(19) United States
(12) Patent Application Publication
(10) Pub. No.: US 2024/0101629 A1
(43) Pub. Date: Mar. 28, 2024

(54) USE OF INTERLEUKIN-7 FOR THE TREATMENT OF CORONAVIRUS

(71) Applicant: NeoImmuneTech, Inc., Rockville, MD (US)

(72) Inventors: Donghoon CHOI, Seongnam-si (KR); Sun-Kyoung IM, Pohang-si (KR); Seungtae BAEK, Pohang-si (KR)

(73) Assignee: NeoImmuneTech, Inc., Rockville, MD (US)

(21) Appl. No.: 18/251,474

(22) PCT Filed: Nov. 2, 2021
(86) PCT No.: PCT/US2021/057744

§ 371 Date: May 2, 2023

Related U.S. Application Data
(60) Provisional application No. 63/108,778, filed on Nov. 2, 2020.

(51) Int. Cl. C07K 14/54; A61P 37/02

(52) U.S. Cl. CPC C07K 14/5418; A61P 37/02; C07K 2319/31

(57) ABSTRACT

The present disclosure relates to methods of treating a disease or disorder associated with a coronavirus infection (e.g., SARS-CoV-19) in a subject, comprising administering an IL-7 protein to the subject. In some aspects, the IL-7 protein is a long-acting IL-7 protein.

### CROSS-REFERENCE TO RELATED APPLICATIONS

This PCT application claims the priority benefit of U.S. Provisional Application No. 63/108,778, filed Nov. 2, 2020, which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 4241_022PC01_SequenceListing_ST25.txt; Size: 83,398 bytes; and Date of Creation: Nov. 2, 2021) filed with the application is herein incorporated by reference in its entirety.

### BACKGROUND OF THE DISCLOSURE

Coronaviruses are positive-stranded RNA viruses that are known to cause diseases in animals and in humans. In humans, coronaviruses cause respiratory tract infections that are typically mild, such as the common cold. But coronaviruses can also cause much more serious infections such coronavirus-induced severe acute respiratory syndrome (SARS). Woo et al., Microbiol. Immunol. 49:899-908 (2005). In March 2020, the World Health Organization declared the outbreak of coronavirus disease 2019 (COVID-19) a pandemic. Like the severe acute respiratory syndrome (SARS) outbreak of 2003, and the Middle East respiratory syndrome (MERS) outbreaks of 2012, 2015, and 2018, COVID-19 has caused serious illness and death around the world.

Currently, there is no approved vaccine and no specific treatment that has garnered approval of the scientific and medical community, although several vaccine and antiviral approaches are being investigated. Accordingly, there exists a need for treatment and prophylaxis of COVID-19.

### SUMMARY OF THE DISCLOSURE

Provided herein is a method of treating a disease or disorder associated with a coronavirus infection in a subject in need thereof, comprising administering to the subject an effective amount of an interleukin-7 (IL-7) protein, wherein the IL-7 protein is a long-acting IL-7 protein. In some aspects, the coronavirus infection is associated with a mild disease as defined by the WHO Ordinal Scale (i.e., <4). In some aspects, the coronavirus infection is associated with a severe disease as defined by the WHO Ordinal Scale (i.e., 5-7). In certain aspects, the subject does not exhibit severe hypoxic respiratory failure.

In some aspects, an absolute lymphocyte count (ALC) is increased in the subject after the administration compared to a corresponding value in a reference subject (e.g., the subject prior to the IL-7 protein administration or a corresponding subject that did not receive the IL-7 protein). In certain aspects, the ALC is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference subject.

In some aspects, a total number of an immune cell is increased in the subject after the administration compared to a corresponding value in a reference subject (e.g., the subject prior to the IL-7 protein administration or a corresponding subject that did not receive the IL-7 protein). In certain aspects, the total number of the immune cell is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference subject. In some aspects, the immune cell comprises a CD4⁺ T cell, CD8⁺ T cell, NK cell, B cell, mucosal associated invariant T (MAIT) cell, innate lymphoid cells (ILCs), or combinations thereof.

In some aspects, a viral load is decreased in the subject after the administration compared to a corresponding value in a reference subject (e.g., the subject prior to the IL-7 protein administration or a corresponding subject that did not receive the IL-7 protein). In certain aspects, the viral load is decreased by at least about 5%, at least about 10%, at least about 20%, at least about 30%, at least about 40%, at least about 50%, at least about 60%, at least about 70%, at least about 80%, at least about 90%, or about 100% compared to a corresponding value in the reference subject.

Also disclosed herein is a method of treating and/or reducing a lymphopenia in a subject suffering from a coronavirus infection, comprising administering to the subject an effective amount of an interleukin-7 (IL-7) protein, wherein the IL-7 protein is a long-acting IL-7 protein. In some aspects, the coronavirus infection is associated with a mild disease as defined by the WHO Ordinal Scale (i.e., <4). In some aspects, the coronavirus infection is associated with a severe disease as defined by the WHO Ordinal Scale (i.e., 5-7). In certain aspects, the subject does not exhibit a severe hypoxic respiratory failure.

In some aspects, a lymphopenia that can be treated and/or reduced using the methods disclosed herein is characterized by a total lymphocyte count that is less than by at least about 5%, at least about 10%, at least about 20%, at least about 30%, at least about 40%, at least about 50%, at least about 60%, at least about 70%, at least about 80%, at least about 90%, or at least about 100% compared to a corresponding value in a reference subject (e.g., subject who does not have a coronavirus). In certain aspects, the lymphopenia is characterized by a circulating blood total lymphocyte count of less than about 1,500 lymphocytes/μL, less than about 1,000 lymphocytes/μL, less than about 800 lymphocytes/μL, less than about 500 lymphocytes/μL, less than about 200 lymphocytes/μL, less than about 100 lymphocytes/μL, or less than about 50 lymphocytes/μL.

In some aspects, the lymphopenia is characterized by a decrease in the number of CD4⁺ T cells, CD8⁺ T cells, or both, compared to a corresponding value in a reference subject (e.g., subject who is not suffering from a coronavirus infection). In certain aspects, the number of CD4⁺ T cells is decreased by at least about 5%, at least about 10%, at least about 20%, at least about 30%, at least about 40%, at least about 50%, at least about 60%, at least about 70%, at least about 80%, at least about 90%, or at least about 100% compared to the corresponding value in the reference subject. In certain aspects, the number of CD8⁺ T cells is decreased by at least about 5%, at least about 10%, at least about 20%, at least about 30%, at least about 40%, at least about 50%, at least about 60%, at least about 70%, at least about 80%, at least about 90%, or at least about 100% compared to the corresponding value in the reference subject. In some aspects, the number of both CD4⁺ T cells and CD8⁺ T cells are decreased by at least about 5%, at least about 10%, at least about 20%, at least about 30%, at least about 40%, at least about 50%, at least about 60%, at least about 70%, at least about 80%, at least about 90%, or at least about 100% compared to the corresponding value in the reference subject.

In some aspects, the total lymphocyte count in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, after the administration of the IL-7 protein. In some aspects, the number of CD4⁺ T cells in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, after the administration of the IL-7 protein. In some aspects, the number of CD8⁺ T cells in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, after the administration of the IL-7 protein. In some aspects, the number of both CD4⁺ T cells and CD8⁺ T cells in the subject is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, after the administration of the IL-7 protein.

Also provided herein is a method of increasing a survival of an immune cell in a subject suffering from a coronavirus infection, comprising administering to the subject an effective amount of an interleukin-7 (IL-7) protein, wherein the IL-7 protein is a long-acting IL-7 protein. In some aspects, the coronavirus infection is associated with a mild disease as defined by the WHO Ordinal Scale (i.e., <4). In some aspects, the coronavirus infection is associated with a severe disease as defined by the WHO Ordinal Scale (i.e., 5-7). In certain aspects, the subject does not exhibit a severe hypoxic respiratory failure.

In some aspects, the survival of the immune cell is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to a corresponding value in a reference subject (e.g., the subject prior to the IL-7 protein administration or a corresponding subject that did not receive the IL-7 protein). In certain aspects, the increase in survival is associated with an increase in the number of the immune cell in the subject, compared to a corresponding value in a reference subject (e.g., the subject prior to the IL-7 protein administration or a corresponding subject that did not receive the IL-7 protein). In some aspects, the number of immune cell is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference subject. In certain aspects, the immune cell comprises a CD4+ T cell, CD8+ T cell, NK cell, B cell, mucosal associated invariant T (MAIT) cell, innate lymphoid cells (ILCs), or combinations thereof.

In any of the methods disclosed herein, in some aspects, the subject exhibits a lymphopenia prior to the administration of the IL-7 protein. In certain aspects, the lymphopenia is associated with an absolute lymphocyte count of about ≤1,000 cells/mm³.

In some aspects, any of the methods disclosed herein further comprises administering one or more additional therapeutic agents to the subject. In certain aspects, the IL-7 protein and the one or more additional therapeutic agents are administered concurrently. In some aspects, the IL-7 protein and the one or more additional therapeutic agents are administered sequentially.

In some aspects, the one or more additional therapeutic agents comprise a standard care of treatment, antiprotozoal agent (e.g., chloroquine or hydroxychloroquine (with or without azithromycin)), antiparasitic agent (e.g., ivermectin), antibiotic agent (e.g., azithromycin), protease inhibitor (e.g., lopinavir/ritonavir or darunavir/cobicistat), immune-based therapy, adjunctive therapy (e.g., antithrombotic therapy), vitamins (e.g., vitamin C (ascorbic acid) and vitamin D), zinc supplementation, or combinations thereof. In some aspects, the standard of care treatment comprises an intravenous fluid, hemodynamic management, transfusion of blood and blood products, renal replacement therapy (RRT) (e.g., in patients with acute kidney injury), corticosteroids (e.g., dexamethasone, prednisone, methylprednisolone, or hydrocortisone) (e.g., in hospitalized patients requiring mechanical ventilation or supplemental oxygenation), antibiotic, antiviral (e.g., remdesivir), antibacterial agent, antiemetic, antiparasitics, oxygenation, and ventilation, and combinations thereof.

In some aspects, the immune-based therapy comprises blood-derived products, immunomodulatory agents, or both. In certain aspects, the blood-derived products comprise COVID-19 convalescent plasma, SARS-CoV-2 immunoglobulins, non-SARS-CoV-2-specific intravenous immunoglobulins (IVIG), mesenchymal stem cells, or combinations thereof. In some aspects, the immunomodulatory agents comprise corticosteroids (e.g., dexamethasone, prednisone, methylprednisolone, or hydrocortisone); interleukin-1 inhibitors (e.g., anakinra); interleukin-6 inhibitors, such as anti-IL-6 receptor antibody (e.g., sarilumab or tocilizumab) or anti-IL-6 antibody (e.g., siltuximab); interferons (e.g., interferon beta-1a, interferon beta-1b, or interferon alfa-2b); kinase inhibitors, such as Bruton's tyrosine kinase inhibitors (e.g., acalabrutinib, ibrutinib, zanubrutinib) or Janus kinase inhibitors (e.g., baricitinib, ruxolitinib, tofacitinib); or combinations thereof.

In the methods disclosed herein, in some aspects, the coronavirus comprises a SARS-CoV-1, SARS-CoV-2 (COVID-19), MERS-CoV, including mutants and variants thereof, or combinations thereof.

In any of the present methods, in some aspects, the IL-7 protein is administered at a dose between about 20 μg/kg and about 600 μg/kg. In certain aspects, the IL-7 protein is administered at a dose of about 20 μg/kg, about 60 μg/kg, about 120 μg/kg, about 240 μg/kg, about 480 μg/kg, or about 600 μg/kg. In some aspects, the IL-7 protein is administered at a dose of about 60 μg/kg, about 120 μg/kg, about 240 μg/kg. or combinations thereof.

In some aspects, the IL-7 protein is administered at a dose greater than about 600 μg/kg, greater than about 700 μg/kg, greater than about 800 μg/kg, greater than about 900 g/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg. In certain aspects, the IL-7 protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg. In some aspects, the IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, the IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 ag/kg, about 950 μg/kg, about 960 μg/kg, about 980 μg/kg, about 1,000 μg/kg, about 1,100 μg/kg, about 1200 μg/kg, about 1,300 μg/kg, about 1,400 μg/kg, about 1,440 μg/kg, about 1,500 μg/kg, about 1,600 ag/kg, about 1,700 μg/kg, about 1,800 μg/kg, about 1,900 μg/kg, or about 2,000 μg/kg.

In any of the methods disclosed herein, in some aspects, the IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks. In some aspects, the IL-7 protein is administered to the subject intramuscularly, intravenously, intraperitoneally, intraarterially, intrathecally, intralymphaticly, intralesionally, intracapsularly, intraorbitally, intracardiacly, intradermally, transtracheally, subcutaneously, subcuticularly, intraarticularly, subcapsularly, subarachnoidly, intraspinally, epidurally, intrasternally, or combinations thereof.

In some aspects, an IL-7 protein of a method disclosed herein is not a wild-type IL-7 protein (i.e., has been modified). In certain aspects, the IL-7 protein comprises an oligopeptide consisting of 1 to 10 amino acid residues. In some aspects, the amino acid residues are selected from the group consisting of methionine (M), glycine (G), and both. In some aspects, the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In certain aspects, the oligopeptide is selected from the group consisting of glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine, (GMM) methionine-glycine-glycine (MGG), glycine-methionine-glycine (GMG), glycine-glycine-methionine (GGM), glycine-glycine-glycine (GGG), methionine-methionine-methionine-methionine (MMMM), and combinations thereof. In some aspects, the oligopeptide is methionine-glycine-methionine (MGM).

In some aspects, the IL-7 protein comprises a half-life extending moiety. In some aspects, the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof. In some aspects, the half-life extending moiety is an Fc. In certain aspects, the Fc is a hybrid Fc, comprising a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, the IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6 and 15-25.

### BRIEF DESCRIPTION OF THE FIGURES

FIGS. 1A, 1B, 1C, and 1D show the effect of long-acting IL-7 protein treatment on K18-hACE2 knock-in mice infected with SARS-CoV-2 (COVID-19). FIG. 1A provides a schematic of the study design. Long-acting IL-7 protein was administered to the mice either at 7 days prior to coronavirus infection (“−D7”) or at 6 hours post coronavirus infection (“+6H”). Then, both bodyweight and survival of the animals were monitored up to 14 days post-infection. FIG. 1B provides a table showing the different treatment groups. FIG. 1C show the percent bodyweight loss observed in animals from the different treatment groups. FIG. 1D show the percent survival observed in animals from the different treatment groups.

FIG. 2 provides a schematic of the experimental design of the clinical study described in Example 1.

FIG. 3 provides the study calendar for the clinical study described in Example 1.

### DETAILED DESCRIPTION OF THE DISCLOSURE

The present disclosure is directed to the use of an IL-7 protein to treat a disease or disorder associated with a coronavirus (e.g., SARS-CoV-2) infection. Non-limiting examples of the various aspects are shown in the present disclosure.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, a therapeutic agent described herein can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten. In certain aspects, the antigen comprises a coronavirus (e.g., SARS-CoV-2) antigen. In some aspects, the antigen is derived from a spike (S) protein (e.g., the receptor-binding domain (RBD) of the S protein). In some aspects, the antigen is derived from an envelope (E) protein. In some aspects, the antigen is derived from a membrane (M) protein. In some aspects, the antigen comprises a T cell epitope present on a coronavirus (“T-antigen”).

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The term “nucleic acid molecule,” as used herein, is intended to include DNA molecules and RNA molecules. A nucleic acid molecule can be single-stranded or double-stranded, and can be cDNA.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain aspects, a predicted nonessential amino acid residue in an antibody is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, e.g., as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

As used herein, the term “effector function” refers to a specialized function of a differentiated immune cell. An effector function of a T cell, for example, can be cytolytic activity or helper activity including the secretion of cytokines. An effector function in a naive, memory, or memory-type T cell can also include antigen-dependent proliferation. As used herein, the term “effector function” refers to one or more properties of activated cells (e.g., CD4⁺ T cells and CD8⁺ T cells) that can help eradicate and/or kill (e.g., via cytolysis) a coronavirus-infected cell. Non-limiting examples of effector functions include proliferation, expression of cytolytic molecules (e.g., perforin or granzymes), production of cytokines (e.g., TNF-α, IFN-γ, and/or IL-2), trafficking to infected tissues, or combinations thereof.

As used herein, the term “immune cells” refers to any cells of the immune system that are involved in mediating an immune response. Accordingly, in some aspects, immune cells useful for the present disclosure include those cells that can play a role in the treatment of a disease or disorder associated with a coronavirus (e.g., SARS-CoV-2) infection. Non-limiting examples of such immune cells include a T lymphocyte, (e.g., CD4+ T cells or CD8+ T cells), B lymphocyte, natural killer (NK) cell, macrophage, eosinophil, mast cell, dendritic cell, neutrophil, or combination thereof.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

Coronaviruses are enveloped viruses with a positive-sense single-stranded RNA genome and a nucleocapsid of helical symmetry. The viral envelope consists of a lipid bilayer, in which the membrane (M), envelope (E) and spike (S) structural proteins are anchored. Chen et. al., J Med Virol 92(4): 418-423 (April 2020), which is herein incorporated by reference in its entirety. There are seven strains of human coronaviruses currently known: (i) human coronavirus 229E (HCoV-229E); (ii) human coronavirus OC43 (HCoV-OC43); (iii) severe acute respiratory syndrome coronavirus 1 (SARS-CoV-1); (iv) human coronavirus NL63 (HCoV-NL63, New Haven coronavirus); (v) human coronavirus HKU1; (vi) Middle East respiratory syndrome-related coronavirus (MERS-CoV, also known as novel coronavirus 2012 and HCoV-EMC); and (vii) severe acute respiratory syndrome coronavirus 2 (SARS-CoV-2), also known as 2019-nCoV, novel coronavirus 2019, or COVID19. Unless specified otherwise, the term “coronavirus,” as used herein, comprises all coronaviruses within the family Coronaviridae, such as the human coronaviruses described herein, including all mutants and variants thereof. For instance, in some aspects, an IL-7 protein described herein can be used to treat a subject infected with any of the coronaviruses described herein or known in the art, including all mutants and variants thereof. In some aspects, an IL-7 protein described herein is useful to prevent or treat a SARS-CoV-2 infection (including any symptoms resulting from the infection). In some aspects, an IL-7 protein described herein can be used to prevent or treat an infection with a SARS-CoV-2 variant and/or mutant (including any symptoms resulting from the infection). See, e.g., Mohammadi et al., Braz J Infect Dis 25(4): 101606 (July-August 2021), which is incorporated herein by reference in its entirety. In some aspects, the SARS-CoV-2 variant and/or mutant that can be prevented and/or treated comprises an alpha variant (e.g., B.1.1.7). In some aspects, the SARS-CoV-2 variant and/or mutant that can be prevented and/or treated comprises a beta variant (e.g., B.1.351). In some aspects, the SARS-CoV-2 variant and/or mutant that can be prevented and/or treated using the present disclosure comprises a delta variant (e.g., B.1.617.2; NCCP 43390; hCoV-19/Korea119861/KDCA/2021). In some aspects, the SARS-CoV-2 variant and/or mutant that can be prevented and/or treated using the present disclosure comprises a gamma variant (e.g., P.1; NCCP 43380; hCoV-19/Korea/KDCA95637/2021). In some aspects, the SARS-CoV-2 variant and/or mutant that can be prevented and/or treated comprises a lambda variant (e.g., C.37). In some aspects, the SARS-CoV-2 variant and/or mutant that can be prevented and/or treated comprises the alpha variant, beta variant, gamma variant, delta variant, lambda variant, or any combination thereof. In some aspects, the SARS-CoV-2 variant and/or mutant that can be prevented and/or treated comprises both the delta and gamma variants.

The term “fusion protein” refers to proteins created through the joining of two or more genes that originally coded for separate proteins. Translation of this fusion gene results in a single polypeptide or multiple polypeptides with functional properties derived from each of the original proteins. In some aspects, the two or more genes can comprise a substitution, a deletion, and/or an addition in its nucleotide sequence.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesion).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1: 1).

Additionally, an Fe (native or variant) of the present disclosure can be in the form of having native sugar chains, increased sugar chains, or decreased sugar chains compared to the native form, or may be in a deglycosylated form. The immunoglobulin Fc sugar chains can be modified by conventional methods such as a chemical method, an enzymatic method, and a genetic engineering method using a microorganism. The removal of sugar chains from an Fc fragment results in a sharp decrease in binding affinity to the C1q part of the first complement component C1, and a decrease or loss of ADCC or CDC, thereby not inducing any unnecessary immune responses in vivo. In this regard, an immunoglobulin Fc region in a deglycosylated or aglycosylated form may be more suitable to the object of the present disclosure as a drug carrier. As used herein, the term “deglycosylation” refers to an Fc region in which sugars are removed enzymatically from an Fc fragment. Additionally, the term “aglycosylation” means that an Fc fragment is produced in an unglycosylated form by a prokaryote, and preferably in E. coli.

As used herein, the term “interleukin-7” or “IL-7” refers to IL-7 polypeptides and derivatives and analogs thereof having substantial amino acid sequence identity to wild-type mature mammalian IL-7 proteins and substantially equivalent biological activity, e.g., in standard bioassays or assays of IL-7 receptor binding affinity. Additional disclosure relating to IL-7 proteins that can be used with the present disclosure are provided elsewhere herein.

A “variant” of an IL-7 protein is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, inserted or deleted without abolishing biological activity can be found using computer programs well known in the art, for example software for molecular modeling or for producing alignments. The variant IL-7 proteins included within the present disclosure include IL-7 proteins that retain IL-7 activity. IL-7 polypeptides which also include additions, substitutions or deletions are also included within the present disclosure as long as the proteins retain substantially equivalent biological IL-7 activity. For example, truncations of IL-7 which retain comparable biological activity as the full length form of the IL-7 protein are included within the present disclosure. In some aspects, variant IL-7 proteins also include polypeptides that have at least about 70%, at least about 75%, at least about 80%, at least about 85%, at least about 90%, at least about 91%, at least about 92%, at least about 93%, at least about 93%, at least about 95%, at least about 96%, at least about 97%, at least about 98%, at least about 99%, or more sequence identity with wild-type IL-7.

As used herein, the term “signal sequence,” or equivalently, “signal peptide,” refers to a fragment directing the secretion of a biologically active molecule drug and a fusion protein, and it is cut off after being translated in a host cell. The signal sequence as used herein is a polynucleotide encoding an amino acid sequence initiating the movement of the protein penetrating the endoplasmic reticulum (ER) membrane. Useful signal sequences include an antibody light chain signal sequence, e.g., antibody 14.18 (Gillies et al., J. Immunol. Meth 1989. 125:191-202), an antibody heavy chain signal sequence, e.g., MOPC141 an antibody heavy chain signal sequence (Sakano et al., Nature, 1980.286: 676-683), and other signal sequences know in the art (e.g., see Watson et al., Nucleic Acid Research, 1984.12:5145-5164). The characteristics of signal peptides are well known in the art, and the signal peptides conventionally having 16 to 30 amino acids, but they can include more or less number of amino acid residues. Conventional signal peptides consist of three regions of the basic N-terminal region, a central hydrophobic region, and a more polar C-terminal region.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to a coronavirus infection (including diseases or disorders associated with the infection), an effective amount comprises an amount sufficient to increase the absolute lymphocyte count (ALC) and/or total number of immune cells in a subject. In some aspects, an effective amount comprises an amount sufficient to reduce and/or alleviate one or more symptoms of a disease or disorder associated with a coronavirus infection (e.g., lymphopenia). Non-limiting examples of such symptoms are provided elsewhere in the present disclosure. In some aspects, an effective amount comprises an amount sufficient to increase the proliferation and/or promote the survival of an immune cell in a subject.

The term “dosing frequency” refers to the number of times a therapeutic agent (e.g., an IL-7 protein) is administered to a subject within a specific time period. Dosing frequency can be indicated as the number of doses per a given time, for example, once per day, once a week, or once in two weeks. As used herein, “dosing frequency” is applicable where a subject receives multiple (or repeated) administrations of a therapeutic agent.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.” Non-limiting examples of standard of care include: intravenous fluids, hemodynamic management, transfusions of blood and blood products, renal replacement therapy (RRT) (e.g., in patients with acute kidney injury), corticosteroids (e.g., dexamethasone, prednisone, methylprednisolone, or hydrocortisone) (e.g., in hospitalized patients requiring mechanical ventilation or supplemental oxygenation), antibiotics, antivirals (e.g., remdesivir), antibacterial agents, antiemetics, antiparasitics, oxygenation, and ventilation.

As used herein, the term “dose limiting toxicities” (DLTs) is defined as follows: (i) a serious adverse event (AE) that is at least possibly related to the IL-7 protein administration; (ii) a grade 3 or higher adverse event that is at least possibly related to the IL-7 protein administration (excluding injection site swelling, irritation or discomfort); or a clinically significant lab abnormality that is at least possibly related to the IL-7 protein administration.

As used herein, the term “adverse event” (AE) refers to any unfavorable and generally unintended or undesirable sign (including an abnormal laboratory finding), symptom, or disease associated with the use of a medical treatment. A medical treatment can have one or more associated AEs and each AE can have the same or different level of severity. Unless indicated otherwise, the descriptions and grading scales found in the revised NCI Common Terminology Criteria for Adverse Events (CTCAE) version 5.0 will be utilized in assessing any AEs observed after the administration of the IL-7 protein.

The term “suspected adverse reaction” (SAR), as used herein, refers to any adverse event for which there is a reasonable possibility that the drug caused the adverse event. “Reasonable possibility” means there is evidence to suggest a causal relationship between the drug and the adverse event. “Suspected adverse reaction” implies a lesser degree of certainty about causality than adverse reaction, which means any adverse event caused by a drug (e.g., IL-7 protein).

As used herein, the term “life-threatening adverse event” or “life threatening suspected adverse reaction” refers to any adverse drug event or suspected adverse reaction is considered “life-threatening” if, in the view of the investigator, its occurrence places the patient at immediate risk of death. It does not include an adverse event or suspected adverse reaction that, had it occurred in a more severe form, might have caused death.

As used herein, the term “serious adverse event” (SAE) or “serious suspected adverse reaction” refers to an adverse event or suspected adverse reaction is considered “serious” if, in the view of the investigator, it results in any of the following outcomes: (i) death; (ii) a life-threatening adverse event; (iii) inpatient hospitalization or prolongation of existing hospitalization; (iv) a persistent or significant incapacity or substantial disruption of the ability to conduct normal life functions; (v) a congenital anomaly/birth defect; (vi) any other important medical event that does not fit the criteria above but, based upon appropriate medical judgment, may jeopardize the subject and may require medical or surgical intervention to prevent one of the outcomes listed above; and (vii) combinations thereof.

As used herein, the term “treatment emergent adverse event” refers to any event that begins or worsens or or after the date of first dose of study treatment.

The term “reference,” as used herein, refers to a corresponding subject (e.g., a subject having a coronavirus infection) who did not receive an administration of an IL-7 protein described herein. The term “reference” can also refer to the same coronavirus-infected subject but prior to the administration of the IL-7 protein. In certain aspects, the term “reference” refers to an average of a population of subjects (e.g., subjects having a coronavirus infection).

As used herein, the terms “ug” and “uM” are used interchangeably with “μg” and “μM,” respectively.

Various aspects described herein are described in further detail in the following subsections.

### II. Methods of the Disclosure

The present disclosure is directed to a method for treating a disease or disorder associated with a coronavirus (e.g., SARS-CoV-2) infection in a subject in need thereof, comprising administering to the subject an effective amount of an interleukin-7 (IL-7) protein. In some aspects, the IL-7 protein is a long-acting IL-7 protein. As used herein, the term “long-acting IL-7 protein” refers to an IL-7 protein that is conjugated to a half-life extending moiety, such that the half-life of the IL-7 protein is increased compared to an IL-7 protein that is not conjugated to a half-life extending moiety (e.g., wild-type IL-7 protein). Additional disclosure relating to exemplary IL-7 proteins that can be used with the methods disclosed herein are provided elsewhere in the present disclosure.

In some aspects, treating a disease or disorder associated with a coronavirus (e.g., SARS-CoV-2) infection comprises increasing the absolute lymphocyte count (ALC) and/or total number of immune cells in the subject. Human subjects infected with a coronavirus (e.g., SARS-CoV-2) can often exhibit reduced number of lymphocytes and/or immune cells, compared to a normal subject (e.g., healthy subject). Zhou et al., Lancet 395(10229): 1054-1062 (2020), which is incorporated herein by reference in its entirety.

Accordingly, in some aspects, administering an IL-7 protein disclosed herein to a subject suffering from a coronavirus (e.g., SARS-CoV-2) infection increases the ALC of the subject, compared to a corresponding value in a reference subject (e.g., the subject prior to the IL-7 protein administration or a corresponding subject that did not receive the IL-7 protein). In certain aspects, the ALC is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference subject.

In some aspects, administering the IL-7 protein of the present disclosure to a subject suffering from a coronavirus (e.g., SARS-CoV-2) infection increases the total number of immune cells in the subject, compared to a corresponding value in a reference subject (e.g., the subject prior to the IL-7 protein administration or a corresponding subject that did not receive the IL-7 protein). In certain aspects, the total number of the immune cell is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference subject.

As described herein, an immune cell that is increased in number after the IL-7 protein administration comprises any immune cells that are useful in treating a disease or disorder associated with a coronavirus (e.g., SARS-CoV-2) infection. In some aspects, the immune cell comprises CD4⁺ T cell, CD8⁺ T cell, NK cell, B cell, mucosal associated invariant T (MAIT) cell, innate lymphoid cells (ILCs), or combinations thereof. In certain aspects, the immune cell is a CD4⁺ T cell. In some aspects, the immune cell is a CD8⁺ T cell. In some aspects, the immune cell is a B cell.

Not to be bound by any one theory, in some aspects, the increase in ALC and/or total number of immune cells results in an improved immune response, wherein the improved immune response can treat a disease or disorder associated with a coronavirus (e.g., SARS-CoV-2) infection. For instance, in some aspects, the improved immune response comprises greater number of effector T cells (e.g., cytotoxic CD8⁺ T cells) that are capable of targeting and eradicating coronavirus-infected cells (e.g., effector T cells that are specific for the T-antigen of a coronavirus). In certain aspects, the effector T cells (e.g., cytotoxic CD8+ T cells) have increased effector function (e.g., increased expression of cytolytic molecules, such as Granzyme and perforin). In some aspects, the improved immune response comprises increased production of antibodies (e.g., neutralizing antibodies) against a coronavirus (e.g., SARS-CoV-2) antigen. Non-limiting examples of such antigens include the spike (S) protein (e.g., S1 subunit or S2 subunit), the membrane (M) protein, the envelope (E) protein, and combinations thereof.

As will be apparent to those skilled in the arts, an improved immune response allows for enhanced clearance of the coronavirus (e.g., SARS-CoV-2) from the subject and/or improved clinical outcome. Accordingly, in some aspects, administering an IL-7 protein of the present disclosure can decrease the viral load in the coronavirus-infected subject, compared to a corresponding value in a reference subject (e.g., the subject prior to the IL-7 protein administration or a corresponding subject that did not receive the IL-7 protein). In certain aspects, the viral load is decreased by at least about 5%, at least about 10%, at least about 20%, at least about 30%, at least about 40%, at least about 50%, at least about 60%, at least about 70%, at least about 80%, at least about 90%, or about 100% compared to a corresponding value in the reference subject.

In some aspects, administering an IL-7 protein disclosed herein reduces the viral load of the coronavirus in the subject, compared to a corresponding value in a reference subject (e.g., the subject prior to the IL-7 protein administration or a corresponding subject that did not receive the IL-7 protein). In certain aspects, the viral load is reduced by at least about 5%, at least about 10%, at least about 20%, at least about 30%, at least about 40%, at least about 50%, at least about 60%, at least about 70%, at least about 80%, at least about 90%, or at least about 100%, compared to the corresponding value in the reference subject. In some aspects, administration of the IL-7 protein reduces the viral load in the subject to an undectable level. In some aspects, a subject with an undectable viral load is completely free of the virus (i.e., no longer infected with the virus). In some aspects, a subject with an undectable viral load is still infected with the virus, but the amount of the virus is too low to be measured, e.g., using a viral load test. As will be apparent to those in the art, in some aspects, a subject with reduced or undetectable viral load exhibits fewer symptoms associated with the infection. Non-limiting examples of such symptoms are provided elsewhere in the present disclosure.

The viral load of a subject can be measured using any suitable methods known in the art. In certain aspects, the viral load of a subject having been infected with a coronavirus (e.g., SARS-CoV-2) can be measured using the Kaplan-Meier method and/or Cox proportional hazard model.

Accordingly, in some aspects, administering an IL-7 protein disclosed herein can reduce the severity of the coronavirus (e.g., SARS-CoV-2) infection (including diseases or disorders associated with the infection). The severity of the infection can be determined using any suitable methods known in the art. In certain aspects, the severity of the infection is measured by the World Health Organization (WHO) Ordinal Scale (see Table 1, below). In some aspects, after the IL-7 protein administration, the severity of the infection is less than 3 on the WHO Ordinal Scale. In some aspects, after the IL-7 protein administration, the severity of the infection is less than 2 on the WHO Ordinal Scale. In some aspects, after the IL-7 protein administration, the severity of the infection is less than 2 on the WHO Ordinal Scale. In some aspects, after the IL-7 protein administration, the subject has a WHO Ordinal Scale of 0.

TABLE 1
WHO Ordinal Scale

| Patient State | Descriptor | Score |
|---|---|---|
| Uninfected | No clinical or virological evidence of | 0 |
| | infection | |
| Ambulatory | No limitation of activities | 1 |
| | Limitation of activities | 2 |
| Hospitalized - | No oxygen therapy | 3 |
| Mild Disease | Oxygen by mask or nasal prongs | 4 |
| Hospitalized - | Non-invasive ventilation or high-flow | 5 |
| Severe Disease | oxygen | |
| | Intubation and mechanical ventilation | 6 |
| | Ventilation + additional organ support | 7 |
| Dead | Death | 8 |

In some aspects, administering an IL-7 protein disclosed herein reduces the time to discharge of a subject who has been hospitalized as a result of a coronavirus (e.g., SARS-CoV-2) infection, compared to a corresponding value in a reference subject (e.g., the subject prior to the IL-7 protein administration or a corresponding subject that did not receive the IL-7 protein). In certain aspects, the time to discharge is reduced by at least about 5%, at least about 10%, at least about 20%, at least about 30%, at least about 40%, at least about 50%, at least about 60%, at least about 70%, at least about 80%, at least about 90%, or at least about 100%, compared to the corresponding value in the reference subject.

In some aspects, treating a disease or disorder associated with a coronavirus (e.g., SARS-CoV-2) infection comprises reducing and/or alleviating one or more symptoms associated with the coronavirus (e.g., SARS-CoV-2) infection. Non-limiting examples of such symptoms include: fever, cough, tiredness (fatigue), new loss of taste or smell, shortness of breath or difficulty breathing, muscle or body aches, chills, sore throat, congestion or runny nose, headache, chest pain, nausea or vomiting, diarrhea, or combinations thereof.

In some aspects, a symptom associated with a coronavirus (e.g., SARS-CoV-2) infection that can be treated with the methods disclosed herein comprises a lymphopenia. As described herein, the number of lymphocytes and/or immune cells are often reduced in subjects infected with a coronavirus (e.g., SARS-CoV-2). If the infection is severe (e.g., requiring hospitalization), the infected subject can be lymphopenic. See, e.g., Guan et al., N Engl. J Med 382(19): 1860 (May 2020); Huang et al., Lancet 395(10223): 497-506 (February 2020); Tan et al., Signal Transduct Target Ther 5(1): 33 (March 2020), each of which is incorporated herein by reference in its entirety. As used herein, the terms “lymphopenia” and “lymphocytopenia” are used interchangeably and refer to a condition characterized by abnormally low number of circulating immune cells (e.g., lymphocytes). In some aspects, compared to a normal subject (e.g., healthy individual), a lymphopenic subject has reduced number of T-lymphocytes (“T-lymphopenia”) (e.g., CD4⁺ T cell, CD8⁺ T cell, or both), B-lymphocytes (“B-lymphopenia”), and/or NK cells (“NK lymphopenia”).

Quantitatively, lymphopenia can be described by various cutoffs. In some aspects, a lymphopenic subject (i.e., subject with lymphopenia) has a circulating blood total lymphocyte count that is less than by at least about 5%, at least about 10%, at least about 20%, at least about 30%, at least about 40%, at least about 50%, at least about 60%, at least about 70%, at least about 80%, at least about 90%, or at least about 100% compared to a circulating blood total lymphocyte count in a corresponding subject who does not exhibit a lymphopenia. In some aspects, a subject has lymphopenia if the subject has a circulating blood total lymphocyte count of less than about 1,500 lymphocytes/μL, less than about 1,000 lymphocytes/μL, less than about 800 lymphocytes/μL, less than about 500 lymphocytes/μL, or less than about 200 lymphocytes/μL. In certain aspects, a lymphopenic subject has an absolute lymphocyte count (ALC) of less than about 1,500 cells per cubic millimeter. In some aspects, a lymphopenic subject has an absolute lymphocyte count (ALC) of less than about 1,000 cells per cubic millimeter.

Accordingly, in some aspects, the present disclosure provides a method of treating and/or reducing a lymphopenia in a subject suffering from a coronavirus (e.g., SARS-CoV-2) infection, comprising administering to the subject an effective amount of an IL-7 protein (e.g., disclosed herein).

In some aspects, the ALC of the lymphopenic subject suffering from a coronavirus (e.g., SARS-CoV-2) infection is increased after the administration of the IL-7 protein compared to a corresponding value in a reference subject (e.g., the subject prior to the IL-7 protein administration or a corresponding subject that did not receive the IL-7 protein). In some aspects, the ALC is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference subject.

In some aspects, the total number of an immune cell is increased in the lymphopenic subject suffering from a coronavirus (e.g., SARS-CoV-2) infection after the administration of the IL-7 protein compared to a corresponding value in a reference subject (e.g., the subject prior to the IL-7 protein administration or a corresponding subject that did not receive the IL-7 protein). In certain aspects, the total number of the immune cell is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference subject. In some aspects, the immune cell comprises a CD4⁺ T cell, CD8⁺ T cell, NK cell, B cell, mucosal associated invariant T (MAIT) cell, innate lymphoid cells (ILCs), or combinations thereof.

In some aspects, the immune cell is a CD4⁺ T cell, CD8⁺ T cell, or both.

Accordingly, in some aspects, administering an IL-7 protein to a lymphopenic subject suffering from a coronavirus (e.g., SARS-CoV-2) infection increases the number CD4⁺ T cells compared to a corresponding value in a reference subject (e.g., the subject prior to the IL-7 protein administration or a corresponding subject that did not receive the IL-7 protein). In certain aspects, the number of CD4⁺ T cells in the lymphopenic subject suffering from a coronavirus (e.g., SARS-CoV-2) infection is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference subject. In some aspects, administering the IL-7 protein increases the number of CD8⁺ T cells in the lymphopenic subject suffering from a coronavirus (e.g., SARS-CoV-2) infection compared to a corresponding value in the reference subject. In certain aspects, the number of CD8⁺ T cells in the lymphopenic subject suffering from a coronavirus (e.g., SARS-CoV-2) infection is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference subject. In some aspects, the number of both CD4⁺ T cells and CD8⁺ T cells is increased in the lymphopenic subject suffering from a coronavirus (e.g., SARS-CoV-2) infection after the administration, compared to the corresponding values in the reference subject.

Not to be bound by any one theory, in some aspects, an IL-7 protein disclosed herein can increase the ALC and/or total number of immune cells in a subject suffering from a coronavirus (e.g., SARS-CoV-2) infection by increasing the proliferation and/or promoting the survival of immune cells in the subject. In certain aspects, after the administration of the IL-7 protein, the proliferation of an immune cell in the subject suffering from a coronavirus (e.g., SARS-CoV-2) infection is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to a corresponding value in a reference subject (e.g., the subject prior to the IL-7 protein administration or a corresponding subject that did not receive the IL-7 protein). In some aspects, after the administration of the IL-7 protein, the survival of an immune cell in the subject suffering from a coronavirus (e.g., SARS-CoV-2) infection is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to a corresponding value in a reference subject (e.g., the subject prior to the IL-7 protein administration or a corresponding subject that did not receive the IL-7 protein). In some aspects, after the IL-7 protein administration, both the proliferation and survival of an immune cell is increased, compared to corresponding values in a reference subject (e.g., the subject prior to the IL-7 protein administration or a corresponding subject that did not receive the IL-7 protein).

In some aspects, the increased proliferation and/or survival of the immune cell is associated with an increase in the number of the immune cell in the subject, compared to a corresponding value in a reference subject (e.g., the subject prior to the IL-7 protein administration or a corresponding subject that did not receive the IL-7 protein). In certain aspects, the number of immune cell is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more, compared to the corresponding value in the reference subject.

In some aspects, treating a disease or disorder associated with a coronavirus (e.g., SARS-CoV-2) comprises preventing a bodyweight loss in a subject suffering from a coronavirus (e.g., SARS-CoV-2). In some aspects, treating a disease or disorder associated with a coronavirus (e.g., SARS-CoV-2) comprises reducing a bodyweight loss in a subject suffering from a coronavirus (e.g., SARS-CoV-2). In some aspects, compared to a reference subject (e.g., corresponding subject that did not receive the IL-7 protein administration), a bodyweight loss is reduced by at least about 5%, at least about 10%, at least about 20%, at least about 30%, at least about 40%, at least about 50%, at least about 60%, at least about 70%, at least about 80%, at least about 90%, or at least about 100%. In some aspects, after a coronavirus (e.g., SARS-CoV-2) infection, the bodyweight of a subject that received an IL-7 protein administration is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% of the reference bodyweight (e.g., bodyweight of the subject prior to the coronavirus infection).

Where a subject does exhibit loss in bodyweight after a coronavirus (e.g., SARS-CoV-2) infection, in some aspects, an IL-7 protein described herein can help improve a recovery in bodyweight loss. For instance, as demonstrated herein, in some aspects, the rate of bodyweight recovery (i.e., the time required for the bodyweight to return to normal (e.g., bodyweight of the subject prior to the coronavirus infection)) is increased in a subject treated with the IL-7 protein compared to reference subject (e.g., corresponding subject that did not receive the IL-7 protein administration). In some aspects, compared to the reference subject, the rate of bodyweight recovery is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more.

In some aspects, treating a disease or disorder associated with a coronavirus (e.g., SARS-CoV-2) comprises increasing the survival of a subject suffering from a coronavirus (e.g., SARS-CoV-2) infection. In some aspects, increasing the survival comprises increasing the probability that the subject will not succumb to the coronavirus infection. In some aspects, increasing the survival comprises increasing the time before the subject succumbs to the coronavirus infection. Accordingly, as demonstrate herein, in some aspects, compared to a reference subject (e.g., corresponding subject that did not receive the IL-7 protein administration), the survival of a subject treated with an IL-7 protein described herein is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold or more.

As will be apparent to those skilled in the arts, the methods disclosed herein can be useful for a wide range of subjects infected with a coronavirus (e.g., SARS-CoV-2) infection. In certain aspects, a subject that can be treated with the methods disclosed herein comprises one or more of the following criteria: (i) tested PCR positive for SARS-CoV-2 by nasopharyngeal swab, oropharyngeal swab, or saliva; (ii) mild infection, defined as WHO Ordinal Scale <4 (see Table 1); (iii) severe infection, defined as WHO Ordinal Scale 5-7 (see Table 1); and (iv) ALC <1500 cells/mm³ at the time of treatment. In some aspects, the subject has a mild coronavirus infection. In some aspects, the subject has a severe coronavirus infection. In certain aspects, the subject does not exhibit severe hypoxic respiratory failure. Additional criteria of subjects that can be treated with the present methods are provided elsewhere in the present disclosure (see, e.g., Example 1). In some aspects, a subject that can be treated with the present disclosure comprises a nonhuman animal, such as a rat or a mouse. In some aspects, a subject that can be treated is a human.

As described herein, the present methods can be useful in treating a disease or disorder associated with any type of coronavirus, such as those disclosed herein (including any variants or mutants thereof). In some aspects, the coronavirus comprises any of the known human coronaviruses, which include HCoV-229E, HCoV-OC43, SARS-CoV-1, HCoV-NL63, HKU1, MERS-CoV, SARS-CoV-2 (COVID19), or combinations thereof. In certain aspects, the coronavirus is SARS-CoV-2 (COVID19).

In some aspects, the unit dose (e.g., for human use) of an IL-7 protein disclosed herein can be in the range of 0.001 mg/kg to 10 mg/kg. In certain aspects, the unit dose of an IL-7 protein is in the range of 0.01 mg/kg to 2 mg/kg. In some aspects, the unit dose is in the range of 0.02 mg/kg to 1 mg/kg. The unit dose can vary depending on the subject diseases for treatment and the presence of adverse effects.

In some aspects, an IL-7 protein disclosed herein can be administered to a subject at a weight-based dose. In certain aspects, an IL-7 protein can be administered at a weight-based dose between about 20 μg/kg and about 600 μg/kg. In certain aspects, an IL-7 protein of the present disclosure can be administered at a weight-based dose of about 20 μg/kg, about 60 μg/kg, about 120 μg/kg, about 240 μg/kg, about 360 μg/kg, about 480 μg/kg, or about 600 μg/kg. In some aspects, the IL-7 protein is administered to the subject at a dose of 60 μg/kg. In some aspects, the IL-7 protein is administered to the subject at a dose of 120 μg/kg. In some aspects, the IL-7 protein is administered to the subject at a dose of 240 g/kg.

In some aspects, an IL-7 protein disclosed herein can be administered to a subject at a dose greater than about 600 μg/kg. In certain aspects, an IL-7 protein is administered to a subject at a dose greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, an IL-7 protein of the present disclosure is administered at a dose of between 610 μg/kg and about 1,200 μg/kg, between 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, an IL-7 protein of the present disclosure is administered at a dose of between 610 μg/kg and about 1,200 μg/kg. In certain aspects, an IL-7 protein is administered at a dose of between 650 μg/kg and about 1,200 μg/kg. In some aspects, an IL-7 protein is administered at a dose of between about 700 μg/kg and about 1,200 μg/kg. In certain aspects, an IL-7 protein is administered at a dose of between about 750 μg/kg and about 1,200 μg/kg. In certain aspects, an IL-7 protein is administered at a dose of between about 800 μg/kg and about 1,200 μg/kg. In some aspects, an IL-7 protein is administered at a dose of between about 850 μg/kg and about 1,200 μg/kg. In some aspects, an IL-7 protein is administered at a dose of between about 900 μg/kg and about 1,200 μg/kg. In certain aspects, an IL-7 protein is administered at a dose of between about 950 μg/kg and about 1,200 μg/kg. In some aspects, an IL-7 protein disclosed herein is administered at a dose of between about 1,000 μg/kg and about 1,200 μg/kg. In some aspects, an IL-7 protein is administered at a dose of between about 1,050 μg/kg and about 1,200 μg/kg. In some aspects, an IL-7 protein is administered at a dose of between about 1,100 μg/kg and about 1,200 μg/kg. In some aspects, an IL-7 protein is administered at a dose of between about 1,200 μg/kg and about 2,000 μg/kg. In certain aspects, an IL-7 protein is administered at a dose of between about 1,300 μg/kg and about 2,000 μg/kg. In some aspects, an IL-7 protein is administered at a dose of between about 1,500 μg/kg and about 2,000 μg/kg. In some aspects, an IL-7 protein is administered at a dose of between about 1,700 μg/kg and about 2,000 μg/kg. In certain aspects, an IL-7 protein is administered at a dose of between about 610 μg/kg and about 1,000 μg/kg. In some aspects, an IL-7 protein is administered at a dose of between about 650 μg/kg and about 1,000 μg/kg. In certain aspects, an IL-7 protein is administered at a dose of between about 700 μg/kg and about 1,000 μg/kg. In yet certain aspects, an IL-7 protein is administered at a dose of between about 750 μg/kg and about 1,000 μg/kg. In certain aspects, an IL-7 protein is administered at a dose of between about 800 μg/kg and about 1,000 μg/kg. In some aspects, an IL-7 protein is administered at a dose of between about 850 μg/kg and about 1,000 μg/kg. In some aspects, an IL-7 protein of the present disclosure is administered at a dose of between about 900 μg/kg and about 1,000 μg/kg. In some aspects, an IL-7 protein is administered at a dose of between about 950 μg/kg and about 1,000 μg/kg.

In some aspects, an IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, an IL-7 protein is administered at a dose of between about 700 μg/kg and about 900 μg/kg. In certain aspects, an IL-7 protein is administered at a dose of between about 750 μg/kg and about 950 μg/kg. In certain aspects, an IL-7 protein is administered at a dose of between about 700 μg/kg and about 850 μg/kg. In some aspects, an IL-7 protein is administered at a dose of between about 750 μg/kg and about 850 μg/kg. In some aspects, an IL-7 protein is administered at a dose of between about 700 μg/kg and about 800 μg/kg. In some aspects, an IL-7 protein is administered at a dose of between about 800 μg/kg and about 900 μg/kg. In some aspects, an IL-7 protein is administered at a dose of between about 750 μg/kg and about 850 μg/kg. In certain aspects, an IL-7 protein is administered at a dose of between about 850 μg/kg and about 950 μg/kg.

In some aspects, an IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 g/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

In some aspects, an IL-7 protein is administered at a dose of about 650 μg/kg. In some aspects, an IL-7 protein disclosed herein is administered at a dose of about 680 μg/kg. In some aspects, an IL-7 protein is administered at a dose of about 700 μg/kg. In some aspects, an IL-7 protein is administered at a dose of about 720 μg/kg. In certain aspects, an IL-7 protein is administered at a dose of about 740 μg/kg. In some aspects, an IL-7 protein is administered at a dose of about 750 μg/kg. In some aspects, an IL-7 protein is administered at a dose of about 760 μg/kg. In certain aspects, an IL-7 protein is administered at a dose of about 780 μg/kg. In some aspects, an IL-7 protein is administered at a dose of about 800 μg/kg. In certain aspects, an IL-7 protein is administered at a dose of about 820 μg/kg. In certain aspects, an IL-7 protein is administered at a dose of about 840 μg/kg. In some aspects, an IL-7 protein is administered at a dose of about 850 μg/kg. In certain aspects, an IL-7 protein is administered at a dose of about 860 μg/kg. In some aspects, an IL-7 protein is administered at a dose of about 880 μg/kg. In some aspects, an IL-7 protein is administered at a dose of about 900 μg/kg. In certain aspects, an IL-7 protein is administered at a dose of about 920 μg/kg. In some aspects, an IL-7 protein is administered at a dose of about 940 μg/kg. In certain aspects, an IL-7 protein is administered at a dose of about 950 μg/kg. In some aspects, an IL-7 protein is administered at a dose of about 960 μg/kg. In certain aspects, an IL-7 protein is administered at a dose of about 980 μg/kg. In some aspects, an IL-7 protein is administered at a dose of about 1,000 μg/kg. In certain aspects, an IL-7 protein is administered at a dose of about 1,020 μg/kg. In certain aspects, an IL-7 protein is administered at a dose of about 1,040 μg/kg. In some aspects, an IL-7 protein is administered at a dose of about 1,060 μg/kg. In some aspects, an IL-7 protein is administered at a dose of about 1,080 μg/kg. In some aspects, an IL-7 protein is administered at a dose of about 1,100 μg/kg. In certain aspects, an IL-7 protein is administered at a dose of about 1,120 μg/kg. In certain aspects, an IL-7 protein is administered at a dose of about 1,140 μg/kg. In some aspects, an IL-7 protein is administered at a dose of about 1,160 μg/kg. In some aspects, an IL-7 protein is administered at a dose of about 1,180 μg/kg. In certain aspects, an IL-7 protein is administered at a dose of about 1,200 μg/kg. In certain aspects, an IL-7 protein is administered at a dose of about 1,220 μg/kg. In certain aspects, an IL-7 protein is administered at a dose of about 1,240 μg/kg. In some aspects, an IL-7 protein is administered at a dose of about 1,260 μg/kg. In some aspects, an IL-7 protein is administered at a dose of about 1,280 μg/kg. In some aspects, an IL-7 protein is administered at a dose of about 1,300 μg/kg. In certain aspects, an IL-7 protein is administered at a dose of about 1,320 μg/kg. In certain aspects, an IL-7 protein is administered at a dose of about 1,340 μg/kg. In some aspects, an IL-7 protein is administered at a dose of about 1,360 μg/kg. In some aspects, an IL-7 protein is administered at a dose of about 1,380 μg/kg. In certain aspects, an IL-7 protein is administered at a dose of about 1,400 μg/kg. In certain aspects, an IL-7 protein is administered at a dose of about 1,420 μg/kg. In some aspects, an IL-7 protein is administered at a dose of about 1,440 μg/kg. In some aspects, an IL-7 protein is administered at a dose of about 1,460 μg/kg. In some aspects, an IL-7 protein is administered at a dose of about 1,480 μg/kg. In certain aspects, an IL-7 protein is administered at a dose of about 1,500 μg/kg. In certain aspects, an IL-7 protein is administered at a dose of about 1,520 μg/kg. In certain aspects, an IL-7 protein is administered at a dose of about 1,540 μg/kg. In some aspects, an IL-7 protein is administered at a dose of about 1,560 μg/kg. In some aspects, an IL-7 protein is administered at a dose of about 1,580 μg/kg. In some aspects, an IL-7 protein is administered at a dose of about 1,600 μg/kg. In certain aspects, an IL-7 protein is administered at a dose of about 1,620 μg/kg. In certain aspects, an IL-7 protein is administered at a dose of about 1,640 μg/kg. In some aspects, an IL-7 protein is administered at a dose of about 1,660 μg/kg. In some aspects, an IL-7 protein is administered at a dose of about 1,680 μg/kg. In certain aspects, an IL-7 protein is administered at a dose of about 1,700 μg/kg. In certain aspects, an IL-7 protein is administered at a dose of about 1,720 μg/kg. In certain aspects, an IL-7 protein is administered at a dose of about 1,740 μg/kg. In some aspects, an IL-7 protein is administered at a dose of about 1,760 μg/kg. In some aspects, an IL-7 protein is administered at a dose of about 1,780 μg/kg. In some aspects, an IL-7 protein is administered at a dose of about 1,800 μg/kg. In certain aspects, an IL-7 protein is administered at a dose of about 1,820 μg/kg. In certain aspects, an IL-7 protein is administered at a dose of about 1,840 μg/kg. In some aspects, an IL-7 protein is administered at a dose of about 1,860 μg/kg. In some aspects, an IL-7 protein is administered at a dose of about 1,880 μg/kg. In some aspects, an IL-7 protein is administered at a dose of about 1,900 μg/kg. In certain aspects, an IL-7 protein is administered at a dose of about 1,920 μg/kg. In certain aspects, an IL-7 protein is administered at a dose of about 1,940 μg/kg. In some aspects, an IL-7 protein is administered at a dose of about 1,960 μg/kg. In some aspects, an IL-7 protein is administered at a dose of about 1,980 μg/kg. In certain aspects, an IL-7 protein is administered at a dose of about 2,000 μg/kg.

In some aspects, an IL-7 protein can be administered at a flat dose. In certain aspects, an IL-7 protein can be administered at a flat dose of about 0.25 mg to about 9 mg. In some aspects, an IL-7 protein can be administered at a flat dose of about 0.25 mg, about 1 mg, about 3 mg, about 6 mg, or about 9 mg.

In some aspects, a subject disclosed herein receives a single administration of an IL-7 protein at any of the doses described above. In some aspects, an IL-7 protein disclosed herein is administered to a subject at multiple doses (i.e., repeated administrations). In certain embodiments, an IL-7 protein is administered to the subject at least two times, at least three times, at least four times, at least five times, at least six times, at least seven times, at least eight times, at least nine times, or at least ten times or more. In other embodiments, a subject receives a single administration of the IL-7 protein (e.g., prior to, concurrently, or after a coronavirus infection).

In some aspects, an IL-7 protein disclosed herein is administered to a subject prior to a coronavirus (e.g., SARS-CoV-2) infection. In some aspects, “prior to a coronavirus infection” comprises administering a therapeutic agent (e.g., IL-7 protein described herein) to a subject before the subject has been exposed to the coronavirus. Unless indicated otherwise, “exposure to a coronavirus” (or derivative thereof) occurs when a subject is in close contact with a coronavirus, such that an infection could (but does not necessarily have to) occur. In some aspects, “close contact with a coronavirus” occurs when the subject is within six feet and for at least about 15 minutes of (i) someone who is showing symptoms of a coronavirus infection or (ii) an infected person who shows no symptoms but later tests positive for the coronavirus. In some aspects, “prior to a coronavirus infection” comprises administering a therapeutic agent (e.g., IL-7 protein described herein) to a subject after exposure to the coronavirus (e.g., after close contact) but prior to infection. Whether a subject is infected with a coronavirus can be determined using any suitable methods known in the art (e.g., antigen test and/or PCR).

As is apparent from the present disclosure, administering an IL-7 protein described herein to a subject prior to coronavirus infection has distinct therapeutic values. For instance, in some aspects, if a subject is at a high risk for: (i) contracting a coronavirus infection (e.g., medical worker), (ii) suffering a severe illness from a coronavirus infection (e.g., elderly or immunocompromised individuals), or (iii) both (i) and (ii), then administering an IL-7 protein described herein prior to the infection can help minimize such high risks. Accordingly, in some aspects, administering an IL-7 protein to a subject prior to a coronavirus infection reduces the likelihood that the subject will become infected when exposed to the coronavirus. In some aspects, the likelihood of a coronavirus infection is reduced by at least about 5%, at least about 10%, at least about 20%, at least about 30%, at least about 40%, at least about 50%, at least about 60%, at least about 70%, at least about 80%, at least about 90%, or at least about 100% compared to a reference subject (e.g., corresponding subject who did not receive an administration of the IL-7 protein described herein). In some aspects, administering an IL-7 protein prior to a coronavirus infection can help reduce the severity of an illness resulting from a coronavirus should the subject become infected after the IL-7 administration. In some aspects, the severity of the illness resulting from the coronavirus infection is reduced by at least about 5%, at least about 10%, at least about 20%, at least about 30%, at least about 40%, at least about 50%, at least about 60%, at least about 70%, at least about 80%, at least about 90%, or at least about 100% compared to a reference subject (e.g., corresponding subject who did not receive an administration of the IL-7 protein prior to the infection). In some aspects, reducing the severity of an illness resulting from a coronavirus comprises reducing the number and/or severity of symptoms associated with a coronavirus infection. Non-limiting examples of such symptoms are provided elsewhere in the present disclosure.

In some aspects, an IL-7 protein is administered to the subject at least about one day, at least about two days, at least about three days, at least about four days, at least about five days, at least about six days, at least about one week, at least about two weeks, at least about three weeks, at least about one month, at least about two months, at least about three months, at least about four months, at least about five months, at least about six months, at least about seven months, at least about eight months, at least about nine months, at least about 10 months, at least about 11 months, or at least about 12 months prior to a coronavirus infection. In some aspects, the IL-7 protein is administered to the subject about one week prior to the coronavirus infection. In some aspects, an IL-7 protein (e.g., described herein) is administered to the subject within about six hours, within about 12 hours, within about one day, within about two days, within about three days, within about four days, within about five days, within about six days, within about one week, within about two weeks, within about three weeks, or within about one month after exposure to a coronavirus. In some aspects, an IL-7 protein is administered to the subject within about two days after exposure to a coronavirus.

In some aspects, an IL-7 protein of the present disclosure is administered to a subject after a coronavirus (e.g., SARS-CoV-2) infection. In some aspects, an IL-7 protein is administered to the subject after a coronavirus infection but before the subject exhibits any symptoms associated with the infection. In some aspects, an IL-7 protein is administered to the subject after the subject exhibits one or more symptoms associated with the coronavirus infection. Non-limiting examples of such symptoms are described elsewhere in the present disclosure.

In some aspects, an IL-7 protein is administered to the subject at least about one hour, at least about two hours, at least about three hours, at least about four hours, at least about five hours, at least about six hours, at least about seven hours, at least about eight hours, at least about nine hours, at least about 10 hours, at least about 11 hours, at least about one day, at least about two days, at least about three days, at least about four days, at least about five days, at least about six days, at least about one week, at least about two weeks, at least about three weeks, at least about one month, at least about two months, at least about three months, at least about four months, at least about five months, or at least about six months after the coronavirus infection. In some aspects, an IL-7 protein is administered about six hours after the coronavirus infection.

In some aspects, an IL-7 protein (e.g., described herein) is administered to the subject within about six hours, within about 12 hours, within about one day, within about two days, within about three days, within about four days, within about five days, within about six days, within about one week, within about two weeks, within about three weeks, or within about one month after the onset of one or more symptoms associated with the coronavirus infection. In some aspects, the IL-7 protein is administered to the subject within about 24 hours after the onset of one or more symptoms associated with the coronavirus infection.

Where multiple doses of an IL-7 protein is administered to the subject, in some aspects, at least one dose of the IL-7 protein is administered to the subject prior to a coronavirus (e.g., SARS-CoV-2) infection. In some aspects, all of the doses of the IL-7 protein is administered to the subject prior to the coronavirus infection. In some aspects, at least one dose of the IL-7 protein is administered to the subject after a coronavirus (e.g., SARS-CoV-2) infection. In some aspects, all of the doses of the IL-7 protein is administered to the subject after the coronavirus infection. In some aspects, at least one dose of the IL-7 protein is administered to the subject prior to the coronavirus infection and at least one dose of the IL-7 protein is administered to the subject after the coronavirus infection.

In some aspects, an IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks. In certain aspects, an IL-7 protein is administered at a dosing frequency of about once every 10 days, about once every 20 days, about once every 30 days, about once every 40 days, about once every 50 days, about once every 60 days, about once every 70 days, about once every 80 days, about once every 90 days, or about once every 100 days. In some aspects, the IL-7 protein is administered once in three weeks. In some aspects, the IL-7 protein is administered once a week. In some aspects, the IL-7 protein is administered once in two weeks. In certain aspects, the IL-7 protein is administered once in three weeks. In some aspects, the IL-7 protein is administered once in four weeks. In certain aspects, the IL-7 protein is administered once in six weeks. In certain aspects, the IL-7 protein is administered once in eight weeks. In some aspects, the IL-7 protein is administered once in nine weeks. In certain aspects, the IL-7 protein is administered once in 12 weeks. In some aspects, the IL-7 protein is administered once every 10 days. In certain aspects, the IL-7 protein is administered once every 20 days. In some aspects, the IL-7 protein is administered once every 30 days. In some aspects, the IL-7 protein is administered once every 40 days. In certain aspects, the IL-7 protein is administered once every 50 days. In some aspects, the IL-7 protein is administered once every 60 days. In certain aspects, the IL-7 protein is administered once every 70 days. In some aspects, the IL-7 protein is administered once every 80 days. In certain aspects, the IL-7 protein is administered once every 90 days. In some aspects, the IL-7 protein is administered once every 100 days.

In some aspects, the IL-7 protein is administered twice or more times in an amount of about 720 μg/kg at an interval of about 1 week, about 2 weeks, about 3 weeks, or about 4 weeks. In some aspects, the IL-7 protein is administered twice or more times in an amount of about 840 μg/kg at an interval of about 2 weeks, about 3 weeks, about 4 weeks, or about 5 weeks. In some aspects, the IL-7 protein is administered twice or more times in an amount of about 960 μg/kg at an interval of about 2 weeks, about 3 weeks, about 4 weeks, about 5 weeks, or about 6 weeks. In some aspects, the IL-7 protein is administered twice or more times in an amount of about 1200 μg/kg at an interval of about 3 weeks, about 4 weeks, about 5 weeks, about 6 weeks, about 7 weeks, or about 8 weeks. In some aspects, the IL-7 protein is administered twice or more times in an amount of about 1440 μg/kg at an interval of about 3 weeks, about 4 weeks, about 5 weeks, about 6 weeks, about 7 weeks, about 8 weeks, about 2 months, about 8 weeks, about 10 weeks, about 12 weeks, or about 3 months.

In some aspects, the IL-7 protein is administered at a dose of 60 μg/kg with a dosing frequency of once a week. In some aspects, the IL-7 protein is administered at a dose of 120 μg/kg with a dosing frequency of once a week. In some aspects, the IL-7 protein is administered at a dose of 240 μg/kg with a dosing frequency of once a week. In some aspects, the IL-7 protein is administered at a dose of 480 μg/kg with a dosing frequency of once a week. In some aspects, the IL-7 protein is administered at a dose of 720 μg/kg with a dosing frequency of once a week. In some aspects, the IL-7 protein is administered at a dose of 960 μg/kg with a dosing frequency of once a week. In some aspects, the IL-7 protein is administered at a dose of 1,200 μg/kg with a dosing frequency of once a week. In some aspects, the IL-7 protein is administered at a dose of 1,300 μg/kg with a dosing frequency of once a week. In some aspects, the IL-7 protein is administered at a dose of 1,400 μg/kg with a dosing frequency of once a week. In some aspects, the IL-7 protein is administered at a dose of 1,420 μg/kg with a dosing frequency of once a week. In certain aspects, the IL-7 protein is administered at a dose of 1,440 μg/kg with a dosing frequency of once a week. In certain aspects, the IL-7 protein is administered at a dose of 1,460 μg/kg with a dosing frequency of once a week. In certain aspects, the IL-7 protein is administered at a dose of 1,480 μg/kg with a dosing frequency of once a week. In some aspects, the IL-7 protein is administered at a dose of 1,500 μg/kg with a dosing frequency of once a week. In certain aspects, the IL-7 protein is administered at a dose of 1,600 μg/kg with a dosing frequency of once a week. In some aspects, the IL-7 protein is administered at a dose of 1,700 μg/kg with a dosing frequency of once a week. In some aspects, the IL-7 protein is administered at a dose of 2,000 μg/kg with a dosing frequency of once a week.

In some aspects, the IL-7 protein is administered at a dose of 60 μg/kg with a dosing frequency of once in two weeks. In some aspects, the IL-7 protein is administered at a dose of 120 μg/kg with a dosing frequency of once in two weeks. In some aspects, the IL-7 protein is administered at a dose of 240 μg/kg with a dosing frequency of once in two weeks. In some aspects, the IL-7 protein is administered at a dose of 480 μg/kg with a dosing frequency of once in two weeks. In some aspects, the IL-7 protein is administered at a dose of 720 μg/kg with a dosing frequency of once in two weeks. In some aspects, the IL-7 protein is administered at a dose of 960 μg/kg with a dosing frequency of once in two weeks. In some aspects, the IL-7 protein is administered at a dose of 1,200 μg/kg with a dosing frequency of once in two weeks. In some aspects, the IL-7 protein is administered at a dose of 1,300 μg/kg with a dosing frequency of once in two weeks. In some aspects, the IL-7 protein is administered at a dose of 1,400 μg/kg with a dosing frequency of once in two weeks. In some aspects, the IL-7 protein is administered at a dose of 1,420 μg/kg with a dosing frequency of once in two weeks. In certain aspects, the IL-7 protein is administered at a dose of 1,440 μg/kg with a dosing frequency of once in two weeks. In certain aspects, the IL-7 protein is administered at a dose of 1,460 μg/kg with a dosing frequency of once in two weeks. In certain aspects, the IL-7 protein is administered at a dose of 1,480 μg/kg with a dosing frequency of once in two weeks. In some aspects, the IL-7 protein is administered at a dose of 1,500 μg/kg with a dosing frequency of once in two weeks. In certain aspects, the IL-7 protein is administered at a dose of 1,600 μg/kg with a dosing frequency of once in two weeks. In some aspects, the IL-7 protein is administered at a dose of 1,700 μg/kg with a dosing frequency of once in two weeks. In some aspects, the IL-7 protein is administered at a dose of 2,000 μg/kg with a dosing frequency of once in two weeks.

In some aspects, the IL-7 protein is administered at a dose of 60 μg/kg with a dosing frequency of once in three weeks. In some aspects, the IL-7 protein is administered at a dose of 120 μg/kg with a dosing frequency of once in three weeks. In some aspects, the IL-7 protein is administered at a dose of 240 μg/kg with a dosing frequency of once in three weeks. In some aspects, the IL-7 protein is administered at a dose of 480 μg/kg with a dosing frequency of once in three weeks. In some aspects, the IL-7 protein is administered at a dose of 720 μg/kg with a dosing frequency of once in three weeks. In some aspects, the IL-7 protein is administered at a dose of 960 μg/kg with a dosing frequency of once in three weeks. In some aspects, the IL-7 protein is administered at a dose of 1,200 μg/kg with a dosing frequency of once in three weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In some aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in three weeks. In certain aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In certain aspects, the IL-7 protein is administered at a dose of 1,600 μg/kg with a dosing frequency of once in three weeks. In some aspects, the IL-7 protein is administered at a dose of 1,700 μg/kg with a dosing frequency of once in three weeks. In some aspects, the IL-7 protein is administered at a dose of 2,000 μg/kg with a dosing frequency of once in three weeks.

In some aspects, the IL-7 protein is administered at a dose of 60 μg/kg with a dosing frequency of once in four weeks. In some aspects, the IL-7 protein is administered at a dose of 120 μg/kg with a dosing frequency of once in four weeks. In some aspects, the IL-7 protein is administered at a dose of 240 μg/kg with a dosing frequency of once in four weeks. In some aspects, the IL-7 protein is administered at a dose of 480 μg/kg with a dosing frequency of once in four weeks. In some aspects, the IL-7 protein is administered at a dose of 720 μg/kg with a dosing frequency of once in four weeks. In some aspects, the IL-7 protein is administered at a dose of 960 μg/kg with a dosing frequency of once in four weeks. In some aspects, the IL-7 protein is administered at a dose of 1,200 μg/kg with a dosing frequency of once in four weeks. In some aspects, the IL-7 protein is administered at a dose of 1,300 μg/kg with a dosing frequency of once in four weeks. In some aspects, the IL-7 protein is administered at a dose of 1,400 μg/kg with a dosing frequency of once in four weeks. In some aspects, the IL-7 protein is administered at a dose of 1,420 μg/kg with a dosing frequency of once in four weeks. In certain aspects, the IL-7 protein is administered at a dose of 1,440 μg/kg with a dosing frequency of once in four weeks. In certain aspects, the IL-7 protein is administered at a dose of 1,460 μg/kg with a dosing frequency of once in four weeks. In certain aspects, the IL-7 protein is administered at a dose of 1,480 μg/kg with a dosing frequency of once in four weeks. In some aspects, the IL-7 protein is administered at a dose of 1,500 μg/kg with a dosing frequency of once in four weeks. In certain aspects, the IL-7 protein is administered at a dose of 1,600 μg/kg with a dosing frequency of once in four weeks. In some aspects, the IL-7 protein is administered at a dose of 1,700 μg/kg with a dosing frequency of once in four weeks. In some aspects, the IL-7 protein is administered at a dose of 2,000 μg/kg with a dosing frequency of once in four weeks.

In some aspects, the IL-7 protein is administered at a dose of 60 μg/kg with a dosing frequency of once in five weeks. In some aspects, the IL-7 protein is administered at a dose of 120 μg/kg with a dosing frequency of once in five weeks. In some aspects, the IL-7 protein is administered at a dose of 240 μg/kg with a dosing frequency of once in five weeks. In some aspects, the IL-7 protein is administered at a dose of 480 μg/kg with a dosing frequency of once in five weeks. In some aspects, the IL-7 protein is administered at a dose of 720 μg/kg with a dosing frequency of once in five weeks. In some aspects, the IL-7 protein is administered at a dose of 960 μg/kg with a dosing frequency of once in five weeks. In some aspects, the IL-7 protein is administered at a dose of 1,200 μg/kg with a dosing frequency of once in five weeks. In some aspects, the IL-7 protein is administered at a dose of 1,300 μg/kg with a dosing frequency of once in five weeks. In some aspects, the IL-7 protein is administered at a dose of 1,400 μg/kg with a dosing frequency of once in five weeks. In some aspects, the IL-7 protein is administered at a dose of 1,420 μg/kg with a dosing frequency of once in five weeks. In certain aspects, the IL-7 protein is administered at a dose of 1,440 μg/kg with a dosing frequency of once in five weeks. In certain aspects, the IL-7 protein is administered at a dose of 1,460 μg/kg with a dosing frequency of once in five weeks. In certain aspects, the IL-7 protein is administered at a dose of 1,480 μg/kg with a dosing frequency of once in five weeks. In some aspects, the IL-7 protein is administered at a dose of 1,500 μg/kg with a dosing frequency of once in five weeks. In certain aspects, the IL-7 protein is administered at a dose of 1,600 μg/kg with a dosing frequency of once in five weeks. In some aspects, the IL-7 protein is administered at a dose of 1,700 μg/kg with a dosing frequency of once in five weeks. In some aspects, the IL-7 protein is administered at a dose of 2,000 μg/kg with a dosing frequency of once in five weeks.

In some aspects, the IL-7 protein is administered at a dose of 60 μg/kg with a dosing frequency of once in six weeks. In some aspects, the IL-7 protein is administered at a dose of 120 μg/kg with a dosing frequency of once in six weeks. In some aspects, the IL-7 protein is administered at a dose of 240 μg/kg with a dosing frequency of once in six weeks. In some aspects, the IL-7 protein is administered at a dose of 480 μg/kg with a dosing frequency of once in six weeks. In some aspects, the IL-7 protein is administered at a dose of 720 μg/kg with a dosing frequency of once in six weeks. In some aspects, the IL-7 protein is administered at a dose of 960 μg/kg with a dosing frequency of once in six weeks. In some aspects, the IL-7 protein is administered at a dose of 1,200 μg/kg with a dosing frequency of once in six weeks. In some aspects, the IL-7 protein is administered at a dose of 1,300 μg/kg with a dosing frequency of once in six weeks. In some aspects, the IL-7 protein is administered at a dose of 1,400 μg/kg with a dosing frequency of once in six weeks. In some aspects, the IL-7 protein is administered at a dose of 1,420 μg/kg with a dosing frequency of once in six weeks. In certain aspects, the IL-7 protein is administered at a dose of 1,440 μg/kg with a dosing frequency of once in six weeks. In certain aspects, the IL-7 protein is administered at a dose of 1,460 μg/kg with a dosing frequency of once in six weeks. In certain aspects, the IL-7 protein is administered at a dose of 1,480 μg/kg with a dosing frequency of once in six weeks. In some aspects, the IL-7 protein is administered at a dose of 1,500 μg/kg with a dosing frequency of once in six weeks. In certain aspects, the IL-7 protein is administered at a dose of 1,600 μg/kg with a dosing frequency of once in six weeks. In some aspects, the IL-7 protein is administered at a dose of 1,700 μg/kg with a dosing frequency of once in six weeks. In some aspects, the IL-7 protein is administered at a dose of 2,000 μg/kg with a dosing frequency of once in six weeks.

In some aspects, the IL-7 protein is administered at a dose of 60 μg/kg with a dosing frequency of once in seven weeks. In some aspects, the IL-7 protein is administered at a dose of 120 μg/kg with a dosing frequency of once in seven weeks. In some aspects, the IL-7 protein is administered at a dose of 240 μg/kg with a dosing frequency of once in seven weeks. In some aspects, the IL-7 protein is administered at a dose of 480 μg/kg with a dosing frequency of once in seven weeks. In some aspects, the IL-7 protein is administered at a dose of 720 μg/kg with a dosing frequency of once in seven weeks. In some aspects, the IL-7 protein is administered at a dose of 960 μg/kg with a dosing frequency of once in seven weeks. In some aspects, the IL-7 protein is administered at a dose of 1,200 μg/kg with a dosing frequency of once in seven weeks. In some aspects, the IL-7 protein is administered at a dose of 1,300 μg/kg with a dosing frequency of once in seven weeks. In some aspects, the IL-7 protein is administered at a dose of 1,400 μg/kg with a dosing frequency of once in seven weeks. In some aspects, the IL-7 protein is administered at a dose of 1,420 μg/kg with a dosing frequency of once in seven weeks. In certain aspects, the IL-7 protein is administered at a dose of 1,440 μg/kg with a dosing frequency of once in seven weeks. In certain aspects, the IL-7 protein is administered at a dose of 1,460 μg/kg with a dosing frequency of once in seven weeks. In certain aspects, the IL-7 protein is administered at a dose of 1,480 μg/kg with a dosing frequency of once in seven weeks. In some aspects, the IL-7 protein is administered at a dose of 1,500 μg/kg with a dosing frequency of once in seven weeks. In certain aspects, the IL-7 protein is administered at a dose of 1,600 μg/kg with a dosing frequency of once in seven weeks. In some aspects, the IL-7 protein is administered at a dose of 1,700 μg/kg with a dosing frequency of once in seven weeks. In some aspects, the IL-7 protein is administered at a dose of 2,000 μg/kg with a dosing frequency of once in seven weeks.

In some aspects, the IL-7 protein is administered at a dose of 60 μg/kg with a dosing frequency of once in eight weeks. In some aspects, the IL-7 protein is administered at a dose of 120 μg/kg with a dosing frequency of once in eight weeks. In some aspects, the IL-7 protein is administered at a dose of 240 μg/kg with a dosing frequency of once in eight weeks. In some aspects, the IL-7 protein is administered at a dose of 480 μg/kg with a dosing frequency of once in eight weeks. In some aspects, the IL-7 protein is administered at a dose of 720 μg/kg with a dosing frequency of once in eight weeks. In some aspects, the IL-7 protein is administered at a dose of 960 μg/kg with a dosing frequency of once in eight weeks. In some aspects, the IL-7 protein is administered at a dose of 1,200 μg/kg with a dosing frequency of once in eight weeks. In some aspects, the IL-7 protein is administered at a dose of 1,300 μg/kg with a dosing frequency of once in eight weeks. In some aspects, the IL-7 protein is administered at a dose of 1,400 μg/kg with a dosing frequency of once in eight weeks. In some aspects, the IL-7 protein is administered at a dose of 1,420 μg/kg with a dosing frequency of once in eight weeks. In certain aspects, the IL-7 protein is administered at a dose of 1,440 μg/kg with a dosing frequency of once in eight weeks. In certain aspects, the IL-7 protein is administered at a dose of 1,460 μg/kg with a dosing frequency of once in eight weeks. In certain aspects, the IL-7 protein is administered at a dose of 1,480 μg/kg with a dosing frequency of once in eight weeks. In some aspects, the IL-7 protein is administered at a dose of 1,500 μg/kg with a dosing frequency of once in eight weeks. In certain aspects, the IL-7 protein is administered at a dose of 1,600 μg/kg with a dosing frequency of once in eight weeks. In some aspects, the IL-7 protein is administered at a dose of 1,700 μg/kg with a dosing frequency of once in eight weeks. In some aspects, the IL-7 protein is administered at a dose of 2,000 μg/kg with a dosing frequency of once in eight weeks.

In some aspects, the IL-7 protein is administered at a dose of 60 μg/kg with a dosing frequency of once in nine weeks. In some aspects, the IL-7 protein is administered at a dose of 120 μg/kg with a dosing frequency of once in nine weeks. In some aspects, the IL-7 protein is administered at a dose of 240 μg/kg with a dosing frequency of once in nine weeks. In some aspects, the IL-7 protein is administered at a dose of 480 μg/kg with a dosing frequency of once in nine weeks. In some aspects, the IL-7 protein is administered at a dose of 720 μg/kg with a dosing frequency of once in nine weeks. In some aspects, the IL-7 protein is administered at a dose of 960 μg/kg with a dosing frequency of once in nine weeks. In some aspects, the IL-7 protein is administered at a dose of 1,200 μg/kg with a dosing frequency of once in nine weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In some aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in nine weeks. In certain aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In certain aspects, the IL-7 protein is administered at a dose of 1,600 μg/kg with a dosing frequency of once in three weeks. In some aspects, the IL-7 protein is administered at a dose of 1,700 μg/kg with a dosing frequency of once in nine weeks. In some aspects, the IL-7 protein is administered at a dose of 2,000 μg/kg with a dosing frequency of once in nine weeks.

In some aspects, the IL-7 protein is administered at a dose of 60 μg/kg with a dosing frequency of once in 10 weeks. In some aspects, the IL-7 protein is administered at a dose of 120 μg/kg with a dosing frequency of once in 10 weeks. In some aspects, the IL-7 protein is administered at a dose of 240 μg/kg with a dosing frequency of once in 10 weeks. In some aspects, the IL-7 protein is administered at a dose of 480 μg/kg with a dosing frequency of once in 10 weeks. In some aspects, the IL-7 protein is administered at a dose of 720 μg/kg with a dosing frequency of once in 10 weeks. In some aspects, the IL-7 protein is administered at a dose of 960 μg/kg with a dosing frequency of once in 10 weeks. In some aspects, the IL-7 protein is administered at a dose of 1,200 μg/kg with a dosing frequency of once in 10 weeks. In some aspects, the IL-7 protein is administered at a dose of 1,300 μg/kg with a dosing frequency of once in 10 weeks. In some aspects, the IL-7 protein is administered at a dose of 1,400 μg/kg with a dosing frequency of once in 10 weeks. In some aspects, the IL-7 protein is administered at a dose of 1,420 μg/kg with a dosing frequency of once in 10 weeks. In certain aspects, the IL-7 protein is administered at a dose of 1,440 μg/kg with a dosing frequency of once in 10 weeks. In certain aspects, the IL-7 protein is administered at a dose of 1,460 μg/kg with a dosing frequency of once in 10 weeks. In certain aspects, the IL-7 protein is administered at a dose of 1,480 μg/kg with a dosing frequency of once in 10 weeks. In some aspects, the IL-7 protein is administered at a dose of 1,500 μg/kg with a dosing frequency of once in 10 weeks. In certain aspects, the IL-7 protein is administered at a dose of 1,600 μg/kg with a dosing frequency of once in 10 weeks. In some aspects, the IL-7 protein is administered at a dose of 1,700 μg/kg with a dosing frequency of once in 10 weeks. In some aspects, the IL-7 protein is administered at a dose of 2,000 μg/kg with a dosing frequency of once in 10 weeks.

In some aspects, the IL-7 protein is administered at a dose of 60 μg/kg with a dosing frequency of once in 11 weeks. In some aspects, the IL-7 protein is administered at a dose of 120 μg/kg with a dosing frequency of once in 11 weeks. In some aspects, the IL-7 protein is administered at a dose of 240 μg/kg with a dosing frequency of once in 11 weeks. In some aspects, the IL-7 protein is administered at a dose of 480 μg/kg with a dosing frequency of once in 11 weeks. In some aspects, the IL-7 protein is administered at a dose of 720 μg/kg with a dosing frequency of once in 11 weeks. In some aspects, the IL-7 protein is administered at a dose of 960 μg/kg with a dosing frequency of once in 11 weeks. In some aspects, the IL-7 protein is administered at a dose of 1,200 μg/kg with a dosing frequency of once in 11 weeks. In some aspects, the IL-7 protein is administered at a dose of 1,300 μg/kg with a dosing frequency of once in 11 weeks. In some aspects, the IL-7 protein is administered at a dose of 1,400 μg/kg with a dosing frequency of once in 11 weeks. In some aspects, the IL-7 protein is administered at a dose of 1,420 μg/kg with a dosing frequency of once in 11 weeks. In certain aspects, the IL-7 protein is administered at a dose of 1,440 μg/kg with a dosing frequency of once in 11 weeks. In certain aspects, the IL-7 protein is administered at a dose of 1,460 μg/kg with a dosing frequency of once in 11 weeks. In certain aspects, the IL-7 protein is administered at a dose of 1,480 μg/kg with a dosing frequency of once in 11 weeks. In some aspects, the IL-7 protein is administered at a dose of 1,500 μg/kg with a dosing frequency of once in 11 weeks. In certain aspects, the IL-7 protein is administered at a dose of 1,600 μg/kg with a dosing frequency of once in 11 weeks. In some aspects, the IL-7 protein is administered at a dose of 1,700 μg/kg with a dosing frequency of once in 11 weeks. In some aspects, the IL-7 protein is administered at a dose of 2,000 μg/kg with a dosing frequency of once in 11 weeks.

In some aspects, the IL-7 protein is administered at a dose of 60 μg/kg with a dosing frequency of once in 12 weeks. In some aspects, the IL-7 protein is administered at a dose of 120 μg/kg with a dosing frequency of once in 12 weeks. In some aspects, the IL-7 protein is administered at a dose of 240 μg/kg with a dosing frequency of once in 12 weeks. In some aspects, the IL-7 protein is administered at a dose of 480 μg/kg with a dosing frequency of once in 12 weeks. In some aspects, the IL-7 protein is administered at a dose of 720 μg/kg with a dosing frequency of once in 12 weeks. In some aspects, the IL-7 protein is administered at a dose of 960 μg/kg with a dosing frequency of once in 12 weeks. In some aspects, the IL-7 protein is administered at a dose of 1,200 μg/kg with a dosing frequency of once in 12 weeks. In some aspects, the IL-7 protein is administered at a dose of 1,300 μg/kg with a dosing frequency of once in 12 weeks. In some aspects, the IL-7 protein is administered at a dose of 1,400 μg/kg with a dosing frequency of once in 12 weeks. In some aspects, the IL-7 protein is administered at a dose of 1,420 μg/kg with a dosing frequency of once in 12 weeks. In certain aspects, the IL-7 protein is administered at a dose of 1,440 μg/kg with a dosing frequency of once in 12 weeks. In certain aspects, the IL-7 protein is administered at a dose of 1,460 μg/kg with a dosing frequency of once in 12 weeks. In certain aspects, the IL-7 protein is administered at a dose of 1,480 μg/kg with a dosing frequency of once in 12 weeks. In some aspects, the IL-7 protein is administered at a dose of 1,500 μg/kg with a dosing frequency of once in 12 weeks. In certain aspects, the IL-7 protein is administered at a dose of 1,600 μg/kg with a dosing frequency of once in 12 weeks. In some aspects, the IL-7 protein is administered at a dose of 1,700 μg/kg with a dosing frequency of once in 12 weeks. In some aspects, the IL-7 protein is administered at a dose of 2,000 μg/kg with a dosing frequency of once in 12 weeks.

In some aspects, the IL-7 protein is administered at a dose of 60 μg/kg with a dosing frequency of once every 10 days. In some aspects, the IL-7 protein is administered at a dose of 120 μg/kg with a dosing frequency of once every 10 days. In some aspects, the IL-7 protein is administered at a dose of 240 μg/kg with a dosing frequency of once every 10 days. In some aspects, the IL-7 protein is administered at a dose of 480 μg/kg with a dosing frequency of once every 10 days. In some aspects, the IL-7 protein is administered at a dose of 720 μg/kg with a dosing frequency of once every 10 days. In some aspects, the IL-7 protein is administered at a dose of 960 μg/kg with a dosing frequency of once every 10 days. In some aspects, the IL-7 protein is administered at a dose of 1,200 μg/kg with a dosing frequency of once every 10 days. In some aspects, the IL-7 protein is administered at a dose of 1,300 μg/kg with a dosing frequency of once every 10 days. In some aspects, the IL-7 protein is administered at a dose of 1,400 μg/kg with a dosing frequency of once every 10 days. In some aspects, the IL-7 protein is administered at a dose of 1,420 μg/kg with a dosing frequency of once every 10 days. In certain aspects, the IL-7 protein is administered at a dose of 1,440 μg/kg with a dosing frequency of once every 10 days. In certain aspects, the IL-7 protein is administered at a dose of 1,460 μg/kg with a dosing frequency of once every 10 days. In certain aspects, the IL-7 protein is administered at a dose of 1,480 μg/kg with a dosing frequency of once every 10 days. In some aspects, the IL-7 protein is administered at a dose of 1,500 μg/kg with a dosing frequency of once every 10 days. In certain aspects, the IL-7 protein is administered at a dose of 1,600 μg/kg with a dosing frequency of once every 10 days. In some aspects, the IL-7 protein is administered at a dose of 1,700 μg/kg with a dosing frequency of once every 10 days. In some aspects, the IL-7 protein is administered at a dose of 2,000 μg/kg with a dosing frequency of once every 10 days.

In some aspects, the IL-7 protein is administered at a dose of 60 μg/kg with a dosing frequency of once every 20 days. In some aspects, the IL-7 protein is administered at a dose of 120 μg/kg with a dosing frequency of once every 20 days. In some aspects, the IL-7 protein is administered at a dose of 240 μg/kg with a dosing frequency of once every 20 days. In some aspects, the IL-7 protein is administered at a dose of 480 μg/kg with a dosing frequency of once every 20 days. In some aspects, the IL-7 protein is administered at a dose of 720 μg/kg with a dosing frequency of once every 20 days. In some aspects, the IL-7 protein is administered at a dose of 960 μg/kg with a dosing frequency of once every 20 days. In some aspects, the IL-7 protein is administered at a dose of 1,200 μg/kg with a dosing frequency of once every 20 days. In some aspects, the IL-7 protein is administered at a dose of 1,300 μg/kg with a dosing frequency of once every 20 days. In some aspects, the IL-7 protein is administered at a dose of 1,400 μg/kg with a dosing frequency of once every 20 days. In some aspects, the IL-7 protein is administered at a dose of 1,420 μg/kg with a dosing frequency of once every 20 days. In certain aspects, the IL-7 protein is administered at a dose of 1,440 μg/kg with a dosing frequency of once every 20 days. In certain aspects, the IL-7 protein is administered at a dose of 1,460 μg/kg with a dosing frequency of once every 20 days. In certain aspects, the IL-7 protein is administered at a dose of 1,480 μg/kg with a dosing frequency of once every 20 days. In some aspects, the IL-7 protein is administered at a dose of 1,500 μg/kg with a dosing frequency of once every 20 days. In certain aspects, the IL-7 protein is administered at a dose of 1,600 μg/kg with a dosing frequency of once every 20 days. In some aspects, the IL-7 protein is administered at a dose of 1,700 μg/kg with a dosing frequency of once every 20 days. In some aspects, the IL-7 protein is administered at a dose of 2,000 μg/kg with a dosing frequency of once every 20 days.

In some aspects, the IL-7 protein is administered at a dose of 60 μg/kg with a dosing frequency of once every 30 days. In some aspects, the IL-7 protein is administered at a dose of 120 μg/kg with a dosing frequency of once every 30 days. In some aspects, the IL-7 protein is administered at a dose of 240 μg/kg with a dosing frequency of once every 30 days. In some aspects, the IL-7 protein is administered at a dose of 480 μg/kg with a dosing frequency of once every 30 days. In some aspects, the IL-7 protein is administered at a dose of 720 μg/kg with a dosing frequency of once every 30 days. In some aspects, the IL-7 protein is administered at a dose of 960 μg/kg with a dosing frequency of once every 30 days. In some aspects, the IL-7 protein is administered at a dose of 1,200 μg/kg with a dosing frequency of once every 30 days. In some aspects, the IL-7 protein is administered at a dose of 1,300 μg/kg with a dosing frequency of once every 30 days. In some aspects, the IL-7 protein is administered at a dose of 1,400 μg/kg with a dosing frequency of once every 30 days. In some aspects, the IL-7 protein is administered at a dose of 1,420 μg/kg with a dosing frequency of once every 30 days. In certain aspects, the IL-7 protein is administered at a dose of 1,440 μg/kg with a dosing frequency of once every 30 days. In certain aspects, the IL-7 protein is administered at a dose of 1,460 μg/kg with a dosing frequency of once every 30 days. In certain aspects, the IL-7 protein is administered at a dose of 1,480 μg/kg with a dosing frequency of once every 30 days. In some aspects, the IL-7 protein is administered at a dose of 1,500 μg/kg with a dosing frequency of once every 30 days. In certain aspects, the IL-7 protein is administered at a dose of 1,600 μg/kg with a dosing frequency of once every 30 days. In some aspects, the IL-7 protein is administered at a dose of 1,700 μg/kg with a dosing frequency of once every 30 days. In some aspects, the IL-7 protein is administered at a dose of 2,000 μg/kg with a dosing frequency of once every 30 days.

In some aspects, the IL-7 protein is administered at a dose of 60 μg/kg with a dosing frequency of once every 40 days. In some aspects, the IL-7 protein is administered at a dose of 120 μg/kg with a dosing frequency of once every 40 days. In some aspects, the IL-7 protein is administered at a dose of 240 μg/kg with a dosing frequency of once every 40 days. In some aspects, the IL-7 protein is administered at a dose of 480 μg/kg with a dosing frequency of once every 40 days. In some aspects, the IL-7 protein is administered at a dose of 720 μg/kg with a dosing frequency of once every 40 days. In some aspects, the IL-7 protein is administered at a dose of 960 μg/kg with a dosing frequency of once every 40 days. In some aspects, the IL-7 protein is administered at a dose of 1,200 μg/kg with a dosing frequency of once every 40 days. In some aspects, the IL-7 protein is administered at a dose of 1,300 μg/kg with a dosing frequency of once every 40 days. In some aspects, the IL-7 protein is administered at a dose of 1,400 μg/kg with a dosing frequency of once every 40 days. In some aspects, the IL-7 protein is administered at a dose of 1,420 μg/kg with a dosing frequency of once every 40 days. In certain aspects, the IL-7 protein is administered at a dose of 1,440 μg/kg with a dosing frequency of once every 40 days. In certain aspects, the IL-7 protein is administered at a dose of 1,460 μg/kg with a dosing frequency of once every 40 days. In certain aspects, the IL-7 protein is administered at a dose of 1,480 μg/kg with a dosing frequency of once every 40 days. In some aspects, the IL-7 protein is administered at a dose of 1,500 μg/kg with a dosing frequency of once every 40 days. In certain aspects, the IL-7 protein is administered at a dose of 1,600 μg/kg with a dosing frequency of once every 40 days. In some aspects, the IL-7 protein is administered at a dose of 1,700 μg/kg with a dosing frequency of once every 40 days. In some aspects, the IL-7 protein is administered at a dose of 2,000 μg/kg with a dosing frequency of once every 40 days.

In some aspects, the IL-7 protein is administered at a dose of 60 μg/kg with a dosing frequency of once every 50 days. In some aspects, the IL-7 protein is administered at a dose of 120 μg/kg with a dosing frequency of once every 50 days. In some aspects, the IL-7 protein is administered at a dose of 240 μg/kg with a dosing frequency of once every 50 days. In some aspects, the IL-7 protein is administered at a dose of 480 μg/kg with a dosing frequency of once every 50 days. In some aspects, the IL-7 protein is administered at a dose of 720 μg/kg with a dosing frequency of once every 50 days. In some aspects, the IL-7 protein is administered at a dose of 960 μg/kg with a dosing frequency of once every 50 days. In some aspects, the IL-7 protein is administered at a dose of 1,200 μg/kg with a dosing frequency of once every 50 days. In some aspects, the IL-7 protein is administered at a dose of 1,300 μg/kg with a dosing frequency of once every 50 days. In some aspects, the IL-7 protein is administered at a dose of 1,400 μg/kg with a dosing frequency of once every 50 days. In some aspects, the IL-7 protein is administered at a dose of 1,420 μg/kg with a dosing frequency of once every 50 days. In certain aspects, the IL-7 protein is administered at a dose of 1,440 μg/kg with a dosing frequency of once every 50 days. In certain aspects, the IL-7 protein is administered at a dose of 1,460 μg/kg with a dosing frequency of once every 50 days. In certain aspects, the IL-7 protein is administered at a dose of 1,480 μg/kg with a dosing frequency of once every 50 days. In some aspects, the IL-7 protein is administered at a dose of 1,500 μg/kg with a dosing frequency of once every 50 days. In certain aspects, the IL-7 protein is administered at a dose of 1,600 μg/kg with a dosing frequency of once every 50 days. In some aspects, the IL-7 protein is administered at a dose of 1,700 μg/kg with a dosing frequency of once every 50 days. In some aspects, the IL-7 protein is administered at a dose of 2,000 μg/kg with a dosing frequency of once every 50 days.

In some aspects, the IL-7 protein is administered at a dose of 60 μg/kg with a dosing frequency of once every 60 days. In some aspects, the IL-7 protein is administered at a dose of 120 μg/kg with a dosing frequency of once every 60 days. In some aspects, the IL-7 protein is administered at a dose of 240 μg/kg with a dosing frequency of once every 60 days. In some aspects, the IL-7 protein is administered at a dose of 480 μg/kg with a dosing frequency of once every 60 days. In some aspects, the IL-7 protein is administered at a dose of 720 μg/kg with a dosing frequency of once every 60 days. In some aspects, the IL-7 protein is administered at a dose of 960 μg/kg with a dosing frequency of once every 60 days. In some aspects, the IL-7 protein is administered at a dose of 1,200 μg/kg with a dosing frequency of once every 60 days. In some aspects, the IL-7 protein is administered at a dose of 1,300 μg/kg with a dosing frequency of once every 60 days. In some aspects, the IL-7 protein is administered at a dose of 1,400 μg/kg with a dosing frequency of once every 60 days. In some aspects, the IL-7 protein is administered at a dose of 1,420 μg/kg with a dosing frequency of once every 60 days. In certain aspects, the IL-7 protein is administered at a dose of 1,440 μg/kg with a dosing frequency of once every 60 days. In certain aspects, the IL-7 protein is administered at a dose of 1,460 μg/kg with a dosing frequency of once every 60 days. In certain aspects, the IL-7 protein is administered at a dose of 1,480 μg/kg with a dosing frequency of once every 60 days. In some aspects, the IL-7 protein is administered at a dose of 1,500 μg/kg with a dosing frequency of once every 60 days. In certain aspects, the IL-7 protein is administered at a dose of 1,600 μg/kg with a dosing frequency of once every 60 days. In some aspects, the IL-7 protein is administered at a dose of 1,700 μg/kg with a dosing frequency of once every 60 days. In some aspects, the IL-7 protein is administered at a dose of 2,000 μg/kg with a dosing frequency of once every 60 days.

In some aspects, the IL-7 protein is administered at a dose of 60 μg/kg with a dosing frequency of once every 70 days. In some aspects, the IL-7 protein is administered at a dose of 120 μg/kg with a dosing frequency of once every 70 days. In some aspects, the IL-7 protein is administered at a dose of 240 μg/kg with a dosing frequency of once every 70 days. In some aspects, the IL-7 protein is administered at a dose of 480 μg/kg with a dosing frequency of once every 70 days. In some aspects, the IL-7 protein is administered at a dose of 720 μg/kg with a dosing frequency of once every 70 days. In some aspects, the IL-7 protein is administered at a dose of 960 μg/kg with a dosing frequency of once every 70 days. In some aspects, the IL-7 protein is administered at a dose of 1,200 μg/kg with a dosing frequency of once every 70 days. In some aspects, the IL-7 protein is administered at a dose of 1,300 μg/kg with a dosing frequency of once every 70 days. In some aspects, the IL-7 protein is administered at a dose of 1,400 μg/kg with a dosing frequency of once every 70 days. In some aspects, the IL-7 protein is administered at a dose of 1,420 μg/kg with a dosing frequency of once every 70 days. In certain aspects, the IL-7 protein is administered at a dose of 1,440 μg/kg with a dosing frequency of once every 70 days. In certain aspects, the IL-7 protein is administered at a dose of 1,460 μg/kg with a dosing frequency of once every 70 days. In certain aspects, the IL-7 protein is administered at a dose of 1,480 μg/kg with a dosing frequency of once every 70 days. In some aspects, the IL-7 protein is administered at a dose of 1,500 μg/kg with a dosing frequency of once every 70 days. In certain aspects, the IL-7 protein is administered at a dose of 1,600 μg/kg with a dosing frequency of once every 70 days. In some aspects, the IL-7 protein is administered at a dose of 1,700 μg/kg with a dosing frequency of once every 70 days. In some aspects, the IL-7 protein is administered at a dose of 2,000 μg/kg with a dosing frequency of once every 70 days.

In some aspects, the IL-7 protein is administered at a dose of 60 μg/kg with a dosing frequency of once every 80 days. In some aspects, the IL-7 protein is administered at a dose of 120 μg/kg with a dosing frequency of once every 80 days. In some aspects, the IL-7 protein is administered at a dose of 240 μg/kg with a dosing frequency of once every 80 days. In some aspects, the IL-7 protein is administered at a dose of 480 μg/kg with a dosing frequency of once every 80 days. In some aspects, the IL-7 protein is administered at a dose of 720 μg/kg with a dosing frequency of once every 80 days. In some aspects, the IL-7 protein is administered at a dose of 960 μg/kg with a dosing frequency of once every 80 days. In some aspects, the IL-7 protein is administered at a dose of 1,200 μg/kg with a dosing frequency of once every 80 days. In some aspects, the IL-7 protein is administered at a dose of 1,300 μg/kg with a dosing frequency of once every 80 days. In some aspects, the IL-7 protein is administered at a dose of 1,400 μg/kg with a dosing frequency of once every 80 days. In some aspects, the IL-7 protein is administered at a dose of 1,420 μg/kg with a dosing frequency of once every 80 days. In certain aspects, the IL-7 protein is administered at a dose of 1,440 μg/kg with a dosing frequency of once every 80 days. In certain aspects, the IL-7 protein is administered at a dose of 1,460 μg/kg with a dosing frequency of once every 80 days. In certain aspects, the IL-7 protein is administered at a dose of 1,480 μg/kg with a dosing frequency of once every 80 days. In some aspects, the IL-7 protein is administered at a dose of 1,500 μg/kg with a dosing frequency of once every 80 days. In certain aspects, the IL-7 protein is administered at a dose of 1,600 μg/kg with a dosing frequency of once every 80 days. In some aspects, the IL-7 protein is administered at a dose of 1,700 μg/kg with a dosing frequency of once every 80 days. In some aspects, the IL-7 protein is administered at a dose of 2,000 μg/kg with a dosing frequency of once every 80 days.

In some aspects, the IL-7 protein is administered at a dose of 60 μg/kg with a dosing frequency of once every 90 days. In some aspects, the IL-7 protein is administered at a dose of 120 μg/kg with a dosing frequency of once every 90 days. In some aspects, the IL-7 protein is administered at a dose of 240 μg/kg with a dosing frequency of once every 90 days. In some aspects, the IL-7 protein is administered at a dose of 480 μg/kg with a dosing frequency of once every 90 days. In some aspects, the IL-7 protein is administered at a dose of 720 μg/kg with a dosing frequency of once every 90 days. In some aspects, the IL-7 protein is administered at a dose of 960 μg/kg with a dosing frequency of once every 90 days. In some aspects, the IL-7 protein is administered at a dose of 1,200 μg/kg with a dosing frequency of once every 90 days. In some aspects, the IL-7 protein is administered at a dose of 1,300 μg/kg with a dosing frequency of once every 90 days. In some aspects, the IL-7 protein is administered at a dose of 1,400 μg/kg with a dosing frequency of once every 90 days. In some aspects, the IL-7 protein is administered at a dose of 1,420 μg/kg with a dosing frequency of once every 90 days. In certain aspects, the IL-7 protein is administered at a dose of 1,440 μg/kg with a dosing frequency of once every 90 days. In certain aspects, the IL-7 protein is administered at a dose of 1,460 μg/kg with a dosing frequency of once every 90 days. In certain aspects, the IL-7 protein is administered at a dose of 1,480 μg/kg with a dosing frequency of once every 90 days. In some aspects, the IL-7 protein is administered at a dose of 1,500 μg/kg with a dosing frequency of once every 90 days. In certain aspects, the IL-7 protein is administered at a dose of 1,600 μg/kg with a dosing frequency of once every 90 days. In some aspects, the IL-7 protein is administered at a dose of 1,700 μg/kg with a dosing frequency of once every 90 days. In some aspects, the IL-7 protein is administered at a dose of 2,000 μg/kg with a dosing frequency of once every 90 days.

In some aspects, the IL-7 protein is administered at a dose of 60 μg/kg with a dosing frequency of once every 100 days. In some aspects, the IL-7 protein is administered at a dose of 120 μg/kg with a dosing frequency of once every 100 days. In some aspects, the IL-7 protein is administered at a dose of 240 μg/kg with a dosing frequency of once every 100 days. In some aspects, the IL-7 protein is administered at a dose of 480 μg/kg with a dosing frequency of once every 100 days. In some aspects, the IL-7 protein is administered at a dose of 720 μg/kg with a dosing frequency of once every 100 days. In some aspects, the IL-7 protein is administered at a dose of 960 μg/kg with a dosing frequency of once every 100 days. In some aspects, the IL-7 protein is administered at a dose of 1,200 μg/kg with a dosing frequency of once every 100 days. In some aspects, the IL-7 protein is administered at a dose of 1,300 μg/kg with a dosing frequency of once every 100 days. In some aspects, the IL-7 protein is administered at a dose of 1,400 μg/kg with a dosing frequency of once every 100 days. In some aspects, the IL-7 protein is administered at a dose of 1,420 μg/kg with a dosing frequency of once every 100 days. In certain aspects, the IL-7 protein is administered at a dose of 1,440 μg/kg with a dosing frequency of once every 100 days. In certain aspects, the IL-7 protein is administered at a dose of 1,460 μg/kg with a dosing frequency of once every 100 days. In certain aspects, the IL-7 protein is administered at a dose of 1,480 μg/kg with a dosing frequency of once every 100 days. In some aspects, the IL-7 protein is administered at a dose of 1,500 μg/kg with a dosing frequency of once every 100 days. In certain aspects, the IL-7 protein is administered at a dose of 1,600 μg/kg with a dosing frequency of once every 100 days. In some aspects, the IL-7 protein is administered at a dose of 1,700 μg/kg with a dosing frequency of once every 100 days. In some aspects, the IL-7 protein is administered at a dose of 2,000 μg/kg with a dosing frequency of once every 100 days.

An IL-7 protein disclosed herein can be administered to a subject having a coronavirus infection by any relevant route of administration. In some aspects, the IL-7 protein is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In certain aspects, the IL-7 protein is administered intramuscularly.

In some aspects, methods disclosed herein (e.g., administering an IL-7 protein) can be used in combination with one or more additional therapeutic agent. In certain aspects, the one or more additional therapeutic agent comprises a standard of care treatment (e.g., such as those described herein) and/or any other compound suitable for treating (e.g., reducing and/or alleviating one or more symptoms of) a conornavirus infection. Non-limiting examples of such compounds include: antiprotozoal agent (e.g., chloroquine or hydroxychloroquine (with or without azithromycin)), antiparasitic agent (e.g., ivermectin), antibiotic agent (e.g., azithromycin), protease inhibitor (e.g., lopinavir/ritonavir or darunavir/cobicistat), immune-based therapy, adjunctive therapy (e.g., antithrombotic therapy), vitamins (e.g., vitamin C (ascorbic acid) and vitamin D), zinc supplementation, or combinations thereof.

In some aspects, immune-based therapy comprises blood-derived products, immunomodulatory agents, or both. Non-limiting examples of blood-derived products include: COVID-19 convalescent plasma, SARS-CoV-2 immunoglobulins, non-SARS-CoV-2-specific intravenous immunoglobulins (IVIG), mesenchymal stem cells, or combinations thereof. Non-limiting examples of immunomodulatory agents include: corticosteroids (e.g., dexamethasone, prednisone, methylprednisolone, or hydrocortisone); interleukin-1 inhibitors (e.g., anakinra); interleukin-6 inhibitors, such as anti-IL-6 receptor antibody (e.g., sarilumab or tocilizumab) or anti-IL-6 antibody (e.g., situximab); interferons (e.g., interferon beta-1a, interferon beta-1b, or interferon alfa-2b); kinase inhibitors, such as Bruton's tyrosine kinase inhibitors (e.g., acalabrutinib, ibrutinib, zanubrutinib) or Janus kinase inhibitors (e.g., baricitinib, ruxolitinib, tofacitinib); or combinations thereof.

### III. IL-7 Proteins Useful for the Disclosure

Disclosed herein are IL-7 proteins that can be used, e.g., to treat a disease or disorder associated with a coronavirus infection. In some aspects, IL-7 protein useful for the present uses can be wild-type IL-7 or modified IL-7 (i.e., not wild-type IL-7 protein) (e.g., IL-7 variant, IL-7 functional fragment, IL-7 derivative, or any combination thereof, e.g., fusion protein, chimeric protein, etc.) as long as the IL-7 protein contains one or more biological activities of IL-7, e.g., capable of binding to IL-7R, e.g., inducing early T-cell development, promoting T-cell homeostasis. See ElKassar and Gress. J Immunotoxicol. 2010 March; 7(1): 1-7, which is incorporated by reference in its entirety. In some aspects, an IL-7 protein of the present disclosure is not a wild-type IL-7 protein (i.e., comprises one or more modifications). Non-limiting examples of such modifications can include an oligopeptide and/or a half-life extending moiety. See WO 2016/200219, which is herein incorporated by reference in its entirety.

IL-7 binds to its receptor which is composed of the two chains IL-7Rα (CD127), shared with the thymic stromal lymphopoietin (TSLP) (Ziegler and Liu, 2006), and the common 7 chain (CD132) for IL-2, IL-15, IL-9 and IL-21. Whereas 7c is expressed by most hematopoietic cells, IL-7Rα is nearly exclusively expressed on lymphoid cells. After binding to its receptor, IL-7 signals through two different pathways: Jak-Stat (Janus kinase-Signal transducer and activator of transcription) and PI3K/Akt responsible for differentiation and survival, respectively. The absence of IL-7 signaling is responsible for a reduced thymic cellularity as observed in mice that have received an anti-IL-7 neutralizing monoclonal antibody (MAb); Grabstein et al., 1993), in IL-7−/− (von Freeden-Jeffry et al., 1995), IL-7Rα−/− (Peschon et al., 1994; Maki et al., 1996), γc−/− (Malissen et al., 1997), and Jak3−/− mice (Park et al., 1995). In the absence of IL-7 signaling, mice lack T-, B-, and NK-T cells. IL-7α−/− mice (Peschon et al., 1994) have a similar but more severe phenotype than IL-7−/− mice (von Freeden-Jeffry et al., 1995), possibly because TSLP signaling is also abrogated in IL-7α−/− mice. IL-7 is required for the development of γδ cells (Maki et al., 1996) and NK-T cells (Boesteanu et al., 1997).

In some aspects, an IL-7 protein useful for the present disclosure comprises an amino acid sequence as set forth in any one of SEQ ID NOs: 1 to 6. In some aspects, the IL-7 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to a sequence of SEQ ID NOS: 1 to 6.

In some aspects, the IL-7 protein includes a modified IL-7 or a fragment thereof, wherein the modified IL-7 or the fragment retains one or more biological activities of wild-type IL-7. In some aspects, the IL-7 protein can be derived from humans, rats, mice, monkeys, cows, or sheep.

In some aspects, the human IL-7 can have an amino acid sequence represented by SEQ ID NO: 1 (Genbank Accession No. P13232); the rat IL-7 can have an amino acid sequence represented by SEQ ID NO: 2 (Genbank Accession No. P56478); the mouse IL-7 can have an amino acid sequence represented by SEQ ID NO: 3 (Genbank Accession No. P10168); the monkey IL-7 may have an amino acid sequence represented by SEQ ID NO: 4 (Genbank Accession No. NP 001279008); the cow IL-7 can have an amino acid sequence represented by SEQ ID NO: 5 (Genbank Accession No. P26895), and the sheep IL-7 can have an amino acid sequence represented by SEQ ID NO: 6 (Genbank Accession No. Q28540).

In some aspects, an IL-7 protein useful for the present disclosure comprises an IL-7 fusion protein. In certain aspects, an IL-7 fusion protein comprises (i) an oligopeptide and (i) an IL-7 or a variant thereof. In some aspects, the oligopeptide is linked to the N-terminal region of the IL-7 or a variant thereof.

In some aspects, an oligopeptide disclosed herein consists of 1 to 10 amino acids. In certain aspects, an oligopeptide consists of at least 1, at least 2, at least 3, at least 4, at least 5, at least 6, at least 7, at least 8, at least 9, or 10 amino acids. In some aspects, one or more amino acids of an oligopeptide are selected from the group consisting of methionine, glycine, and combinations thereof. In certain aspects, an oligopeptide is selected from the group consisting of methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In certain aspects, the oligopeptide is selected from the group consisting of glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine, (GMM) methionine-glycine-glycine (MGG), glycine-methionine-glycine (GMG), glycine-glycine-methionine (GGM), glycine-glycine-glycine (GGG), methionine-methionine-methionine-methionine (MMMM), and combinations thereof. In some aspects, an oligopeptide is methionine-glycine-methionine (MGM).

In some aspects, an IL-7 fusion protein comprises (i) an IL-7 or a variant thereof, and (ii) a half-life extending moiety. In some aspects, a half-life extending moiety extends the half-life of the IL-7 or variant thereof. In some aspects, a half-life extending moiety is linked to the C-terminal region of an IL-7 or a variant thereof.

In some aspects, an IL-7 fusion protein comprises (i) IL-7 (a first domain), (ii) a second domain that includes an amino acid sequence having 1 to 10 amino acid residues consisting of methionine, glycine, or a combination thereof, e.g., MGM, and (iii) a third domain comprising a half-life extending moiety. In some aspects, the half-life extending moiety can be linked to the N-terminal or the C-terminal of the first domain or the second domain. Additionally, the IL-7 including the first domain and the second domain can be linked to both terminals of the third domain.

Non-limiting examples of half-life extending moieties include: Fc, albumin, an albumin-binding polypeptide, Pro/Ala/Ser (PAS), a C-terminal peptide (CTP) of the R subunit of human chorionic gonadotropin, polyethylene glycol (PEG), long unstructured hydrophilic sequences of amino acids (XTEN), hydroxyethyl starch (HES), an albumin-binding small molecule, and combinations thereof.

In some aspects, a half-life extending moiety is Fc. In certain aspects, Fc is from a modified immunoglobulin in which the antibody-dependent cellular cytotoxicity (ADCC) or complement-dependent cytotoxicity (CDC) weakened due to the modification in the binding affinity with the Fc receptor and/or a complement. In some aspects, the modified immunoglobulin can be selected from the group consisting of IgG1, IgG2, IgG3, IgG4, IgA1, IgA2, IgD, IgE, and a combination thereof. In some aspects, an Fc is a hybrid Fc (“hFc” or “hyFc”), comprising a hinge region, a CH2 domain, and a CH3 domain. In certain aspects, a hinge region of a hybrid Fc disclosed herein comprises a human IgD hinge region. In certain aspects, a CH2 domain of a hybrid Fc comprises a part of human IgD CH2 domain and a part of human IgG4 CH2 domain. In certain aspects, a CH3 domain of a hybrid Fc comprises a part of human IgG4 CH3 domain. Accordingly, in some aspects, a hybrid Fc disclosed herein comprises a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, an Fe disclosed herein can be an Fc variant. As used herein, the term “Fc variant” refers to an Fc which was prepared by substituting a part of the amino acids among the Fc region or by combining the Fc regions of different kinds. The Fc region variant can prevent from being cut off at the hinge region. Specifically, in some aspects, a Fc variant comprises modifications at the 144^{th} amino acid and/or 145^{th} amino acid of SEQ ID NO: 9. In certain aspects, the 144^{th} amino acid (K) and/or the 145^{th} amino acid (K) is substituted with G or S.

In some aspects, an Fc or an Fc variant disclosed herein can be represented by the following formula: N′—(Z1)p-Y-Z2-Z3-Z4-C, wherein:
N′ comprises the N-terminus;
Z1 comprises an amino acid sequence having 5 to 9 consecutive amino acid residues from the amino acid residue at position 98 toward the N-terminal, among the amino acid residues at positions from 90 to 98 of SEQ ID NO: 7;
Y comprises an amino acid sequence having 5 to 64 consecutive amino acid residues from the amino acid residue at position 162 toward the N-terminal, among the amino acid residues at positions from 99 to 162 of SEQ ID NO: 7;
Z2 comprises an amino acid sequence having 4 to 37 consecutive amino acid residues from the amino acid residue at position 163 toward the C-terminal, among the amino acid residues at positions from 163 to 199 of SEQ ID NO: 7;
Z3 comprises an amino acid sequence having 71 to 106 consecutive amino acid residues from the amino acid residue at position 220 toward the N-terminal, among the amino acid residues at positions from 115 to 220 of SEQ ID NO: 8; and
Z4 comprises an amino acid sequence having 80 to 107 consecutive amino acid residues from the amino acid residue at position 221 toward the C-terminal, among the amino acid residues at positions from 221 to 327 of SEQ ID NO: 8.

In some aspects, a Fc region disclosed herein can include the amino acid sequence of SEQ ID NO: 9 (hyFc), SEQ ID NO: 10 (hyFcM1), SEQ ID NO: 11 (hyFcM2), SEQ ID NO: 12 (hyFcM3), or SEQ ID NO: 13 (hyFcM4). In some aspects, the Fc region can include the amino acid sequence of SEQ ID NO: 14 (a non-lytic mouse Fc).

Other non-limiting examples of Fc regions that can be used with the present disclosure are described in U.S. Pat. No. 7,867,491, which is herein incorporated by reference in its entirety.

In some aspects, an IL-7 fusion protein disclosed herein comprises both an oligopeptide and a half-life extending moiety.

In some aspects, an IL-7 protein can be fused to albumin, a variant, or a fragment thereof. Examples of the IL-7-albumin fusion protein can be found at International Application Publication No. WO 2011/124718 A1. In some aspects, an IL-7 protein is fused to a pre-pro-B cell Growth Stimulating Factor (PPBSF), optionally by a flexible linker. See US 2002/0058791A1. In some aspects, an IL-7 protein useful for the disclosure is an IL-7 conformer that has a particular three dimensional structure. See US 2005/0249701 A1. In some aspects, an IL-7 protein can be fused to an Ig chain, wherein amino acid residues 70 and 91 in the IL-7 protein are glycosylated the amino acid residue 116 in the IL-7 protein is non-glycosylated. See U.S. Pat. No. 7,323,549 B2. In some aspects, an IL-7 protein that does not contain potential T-cell epitopes (thereby to reduce anti-IL-7 T-cell responses) can also be used for the present disclosure. See US 2006/0141581 A1. In some aspects, an IL-7 protein that has one or more amino acid residue mutations in carboxy-terminal helix D region can be used for the present disclosure. The IL-7 mutant can act as IL-7R partial agonist despite lower binding affinity for the receptor. See US 2005/0054054A1. Any IL-7 proteins described in the above listed patents or publications are incorporated herein by reference in their entireties.

In addition, non-limiting examples of additional IL-7 proteins useful for the present disclosure are described in U.S. Pat. Nos. 7,708,985, 8,034,327, 8,153,114, 7,589,179, 7,323,549, 7,960,514, 8,338,575, 7,118,754, 7,488,482, 7,670,607, 6,730,512, WO0017362, GB2434578A, WO 2010/020766 A2, WO91/01143, Beq et al., Blood, vol. 114 (4), 816, 23 Jul. 2009, Kang et al., J. Virol. Doi:10.1128/JVI.02768-15, Martin et al., Blood, vol. 121 (22), 4484, May 30, 2013, McBride et al., Acta Oncologica, 34:3, 447-451, Jul. 8, 2009, and Xu et al., Cancer Science, 109: 279-288, 2018, which are incorporated herein by reference in their entireties.

In some aspects, an oligopeptide disclosed herein is directly linked to the N-terminal region of IL-7 or a variant thereof. In some aspects, an oligopeptide is linked to the N-terminal region via a linker. In some aspects, a half-life extending moiety disclosed herein is directly linked to the C-terminal region of IL-7 or a variant thereof. In certain aspects, a half-life extending moiety is linked to the C-terminal region via a linker. In some aspects, a linker is a peptide linker. In certain aspects, a peptide linker comprises a peptide of 10 to 20 amino acid residues consisting of Gly and Ser residues. In some aspects, a linker is an albumin linker. In some aspects, a linker is a chemical bond. In certain aspects, a chemical bond comprises a disulfide bond, a diamine bond, a sulfide-amine bond, a carboxy-amine bond, an ester bond, a covalent bond, or combinations thereof. When the linker is a peptide linker, in some aspects, the connection can occur in any linking region. They may be coupled using a crosslinking agent known in the art. In some aspects, examples of the crosslinking agent can include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

In some aspects, an IL-7 (or variant thereof) portion of IL-7 fusion protein disclosed herein comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 15-20. In certain aspects, an IL-7 (or variant thereof) portion of IL-7 fusion protein disclosed herein comprises the amino acid sequence set forth in SEQ ID NOs: 15-20.

In some aspects, an IL-7 fusion protein comprises: a first domain including a polypeptide having the activity of IL-7 or a similar activity thereof, a second domain comprising an amino acid sequence having 1 to 10 amino acid residues consisting of methionine, glycine, or a combination thereof, and a third domain, which is an Fc region of modified immunoglobulin, coupled to the C-terminal of the first domain.

In some aspects, an IL-7 fusion protein that can be used with the present methods comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 21-25. In certain aspects, an IL-7 fusion protein of the present disclosure comprises the amino acid sequence set forth in SEQ ID NOs: 21-25. In certain aspects, an IL-7 fusion protein disclosed herein comprises the amino acid sequence set forth in SEQ ID NOs: 26 and 27.

### IV. Nucleic Acids, Vectors, Host Cells

Further aspect described herein pertains to one or more nucleic acid molecules that encode a therapeutic agent described herein (e.g., an IL-7 protein). The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In a certain aspects, the nucleic acid is a cDNA molecule. Nucleic acids described herein can be obtained using standard molecular biology techniques known in the art.

Certain nucleic acid molecules disclosed herein are those encoding an IL-7 protein (e.g., disclosed herein). Exemplary nucleic acid sequences encoding an IL-7 protein disclosed herein are set forth in SEQ ID NOs: 29-39.

In some aspects, the present disclosure provides a vector comprising an isolated nucleic acid molecule encoding a therapeutic agent disclosed herein (e.g., an IL-7 protein). In some aspects, a vector can be used for gene therapy.

When used as a gene therapy (e.g., in humans), a nucleic acid encoding a therapeutic agent disclosed herein (e.g., an IL-7 protein) can be administered at a dosage in the range of 0.1 mg to 200 mg. In certain aspects, the dosage is in the range of 0.6 mg to 100 mg. In certain aspects, the dosage is in the range of 1.2 mg to 50 mg.

Suitable vectors for the disclosure include expression vectors, viral vectors, and plasmid vectors. In some aspects, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

As used herein, viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934, each which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

Also encompassed by the present disclosure is a method for making a therapeutic agent disclosed herein (e.g., an IL-7 protein). In some aspects, such a method can comprise expressing the therapeutic agent (e.g., an IL-7 protein) in a cell comprising a nucleic acid molecule encoding the therapeutic agent, e.g., SEQ ID NOs: 29-39. Additional details regarding the method for making an TL-7 protein disclosed herein are provided, e.g., in WO 2016/200219, which is herein incorporated by reference in its entirety. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### V. Pharmaceutical Compositions

Further provided herein are compositions comprising one or more therapeutic agents (e.g., an IL-7 protein and/or a standard care of treatment) having the desired degree of purity in a physiologically acceptable carrier, excipient or stabilizer (Remington's Pharmaceutical Sciences (1990) Mack Publishing Co., Easton, PA). In some aspects, a composition disclosed herein comprises an IL-7 protein. As disclosed herein, such composition can be used in combination with other compositions (e.g., comprising an additional therapeutic agent, e.g., standard care of treatment). In some aspects, a composition disclosed herein can comprise both an TL-7 protein and an additional therapeutic agent (e.g., standard of care treatment).

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a composition disclosed herein comprises one or more additional components selected from: a bulking agent, stabilizing agent, surfactant, buffering agent, or combinations thereof.

Buffering agents useful for the current disclosure can be a weak acid or base used to maintain the acidity (pH) of a solution near a chosen value after the addition of another acid or base. Suitable buffering agents can maximize the stability of the pharmaceutical compositions by maintaining pH control of the composition. Suitable buffering agents can also ensure physiological compatibility or optimize solubility. Rheology, viscosity and other properties can also dependent on the pH of the composition. Common buffering agents include, but are not limited to, a Tris buffer, a Tris-Cl buffer, a histidine buffer, a TAE buffer, a HEPES buffer, a TBE buffer, a sodium phosphate buffer, a MES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picoline buffer, a 4-picoline buffer, a 3,5-lutidine buffer, a 3,4-lutidine buffer, a 2,4-lutidine buffer, a Aces, a diethylmalonate buffer, a N-methylimidazole buffer, a 1,2-dimethylimidazole buffer, a TAPS buffer, a bis-Tris buffer, a L-arginine buffer, a lactate buffer, a glycolate buffer, or combinations thereof.

In some aspects, a composition disclosed herein further comprises a bulking agent. Bulking agents can be added to a pharmaceutical product in order to add volume and mass to the product, thereby facilitating precise metering and handling thereof. Bulking agents that can be used with the present disclosure include, but are not limited to, sodium chloride (NaCl), mannitol, glycine, alanine, or combinations thereof.

In some aspects, a composition disclosed herein can also comprise a stabilizing agent. Non-limiting examples of stabilizing agents that can be used with the present disclosure include: sucrose, trehalose, raffinose, arginine, or combinations thereof.

In some aspects, a composition disclosed herein comprises a surfactant. In certain aspects, the surfactant can be selected from the following: alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, dodecyl dimethylamine oxide, or combinations thereof. In some aspects, the surfactant is polysorbate 20 or polysorbate 80.

In some aspects, an IL-7 protein disclosed herein is formulated in a composition comprising (a) a basal buffer, (b) a sugar, and (c) a surfactant. In certain aspects, the basal buffer comprises histidine-acetate or sodium citrate. In some aspects, the basal buffer is at a concentration of about 10 to about 50 nM. In some aspects, a sugar comprises sucrose, trehalose, dextrose, or combinations thereof. In some aspects, the sugar is present at a concentration of about 2.5 to about 5.0 w/v %. In certain aspects, the surfactant is selected from polysorbate, polyoxyethylene alkyl ether, polyoxyethylene stearate, alkyl sulfates, polyvinyl pyridone, poloxamer, or combinations thereof. In some aspects, the surfactant is at a concentration of about 0.05% to about 6.0 w/v %.

In some aspects, a composition disclosed herein (e.g., comprising an IL-7 protein and/or additional therapeutic agent) further comprises an amino acid. In certain aspects, the amino acid is selected from arginine, glutamate, glycine, histidine, or combinations thereof. In certain aspects, the composition further comprises a sugar alcohol. Non-limiting examples of sugar alcohol includes: sorbitol, xylitol, maltitol, mannitol, or combinations thereof.

In some aspects, an IL-7 protein disclosed herein is formulated in a composition comprising the following: (a) sodium citrate (e.g., about 20 mM), (b) sucrose (e.g., about 5%), (c) sorbitol (e.g., about 1.5%), and (d) Tween 80 (e.g., about 0.05%).

In some aspects, an IL-7 protein of the present disclosure is formulated as described in WO 2017/078385 A1, which is incorporated herein in its entirety.

A pharmaceutical composition can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. Parenteral administration, characterized by either subcutaneous, intramuscular or intravenous injection, is also contemplated herein. In some aspects, an IL-7 protein and an additional therapeutic agent (e.g., standard care of treatment) are administered using the same route of administration. In some aspects, an IL-7 protein and an additional therapeutic agent (e.g., standard care of treatment) are administered using different routes of administration.

Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

A therapeutic agent described herein (e.g., an IL-7 protein) can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in one aspect, have diameters of less than 50 microns, in one aspect less than 10 microns.

A therapeutic agent disclosed herein (e.g., an IL-7 protein) can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957, each of which is herein incorporated by reference in its entirety.

In certain aspects, a pharmaceutical composition comprising a therapeutic agent described herein (e.g., an IL-7 protein) is a lyophilized powder, which can be reconstituted for administration as solutions, emulsions and other mixtures. It can also be reconstituted and formulated as solids or gels. The lyophilized powder is prepared by dissolving an antibody or antigen-binding portion thereof described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium or potassium phosphate or other such buffer known to those of skill in the art at, in one aspect, about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier.

Compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, each of which is incorporated herein by reference in its entirety.

The compositions to be used for in vivo administration can be sterile. This is readily accomplished by filtration through, e.g., sterile filtration membranes.

The following examples are merely illustrative and should not be construed as limiting the scope of this disclosure in any way as many variations and equivalents will become apparent to those skilled in the art upon reading the present disclosure.

### EXAMPLES

### Example 1: Analysis of the Effect of IL-7 Protein Administration on SARS-CoV-2 Infection

To begin assessing the effect on SARS-CoV-2 (COVID-19) infection, a long-acting IL-7 protein (e.g., described herein) was administered to K18-hACE2 knock-in mice and both the weight and survival were monitored at various time points. The specific materials and methods used are described below.

### Mice

Female C57BL/6 K18-hACE2 knock-in mice aged 9-14 weeks were purchased from Jackson Laboratory. The weight of the animals used in the experiment was 18-25 g. Mice were randomly divided into three groups and used for the experiment. All mice were bred in the animal facility of Pohang University of Science and Technology (POSTECH; Pohang-si, Korea), and transferred to the International Vaccine Institute (IVI; Seoul, Korea). All mouse studies were conducted at the Animal Biosafety Level 3 (ABSL-3) facility after being reviewed by Institutional Animal Care and Use Committee (IACUC) and Integrative Safety Committee (ISC) of the IVI (IACUC PN 2021-005, ISC-RA-2021-03-09). Study execution and housing were following guidelines of POSTECH and IVI. Mice were kept in individually ventilated cages with a 12-h light/dark cycle, controlled environmental conditions and under specific-pathogen-free conditions. Food and water were available ad libitum. Only mice with an unobjectionable health status were selected for testing procedures. And euthanasia was performed when the mouse weight decreased by more than 20% according to IACUC regulations.

### IL-7 Treatment

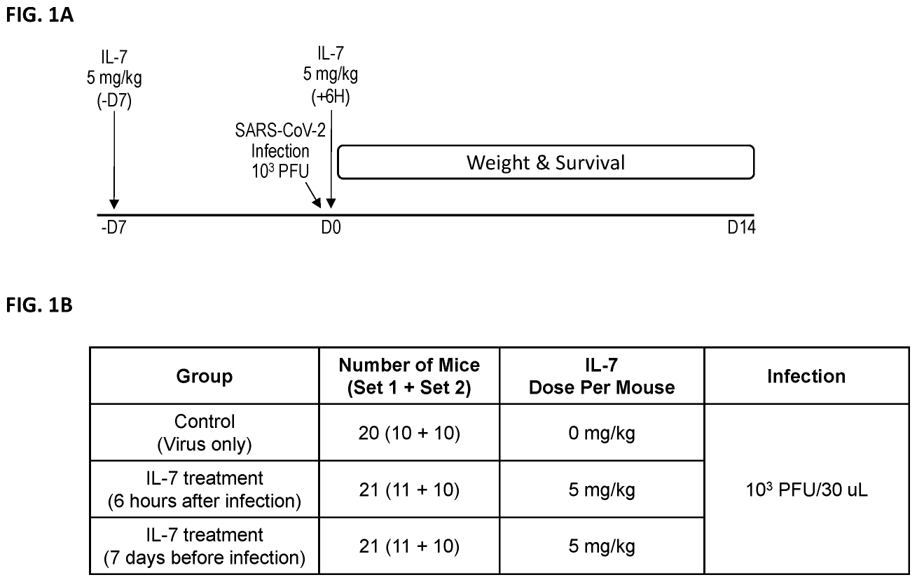

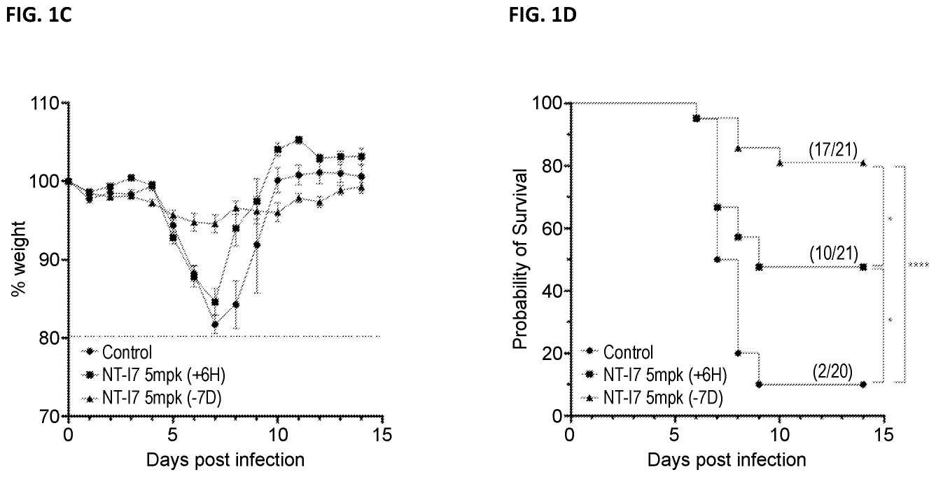

An IL-7 protein (long-acting, such as that described herein) was diluted with formulation buffer and administered to the mice via intramuscular injection at 5 mg/kg per animal. The IL-7 protein was administered either at 7 days before coronavirus infection or 6 hours after coronavirus infection. The control group was administered with formulation buffer 6 hours after infection. See FIGS. 1A and 1B.

### SARS-CoV-2 Virus

SARS-CoV-2 Virus (NCCP 43326 (BetaCoV/Korea/KCDC03/2020)) was acquired from the National Culture Collection for pathogens of Korea Centers for Disease Control and Prevention (KCDC). The virus was cultured using Vero E6 cells.

### Virus Challenge Model

Viral infection was performed on day 0 after anesthesia through intraperitoneal administration of anesthetics. (Anesthetic Ketamine solution composition: 100 mg/kg ketamine (Yuhan, Korea), 12.5 mg/kg rompun (Bayer, Germany), PBS (Thermo, USA)). Specifically, each mouse was infected with 1×10³ PFU/30 uL of SARS-CoV-2/BetaCoV/Korea/KCDC03/2020 virus suspension. Routine animal monitoring, such as body weight and survival checks, was performed daily for 14 days after viral infection.

### Statistical Analysis

Data were analyzed using GraphPad Prism 9 software. For body weight analysis shown in FIG. 1C, continuous variables were tested for the normality and lognormality tests by using the Shapiro-Wilk test. Then a non-parametric test was performed. Kruskal-Wallis one-way ANOVA and Dunn's post hoc tests were used. For multiple comparisons, the mean of each group was compared with the mean of control group. For survival rate analysis shown in FIG. 1D, Kaplan-Meier survival curves were assessed by using the log-rank (Mantel-Cox) test. Data were presented as mean±Standard Error of Mean (SEM). The indications of statistical significance are as follows; ns p≥0.05, * p<0.05, ** p<0.01, *** p<0.001, **** p<0.0001.

### Results

As shown in FIG. 1C, soon after SARS-CoV-2 infection, the control animals (i.e., received formulation buffer alone) exhibited significant weight loss (approximately 20% loss at day 5 post-infection), which returned to normal by about 10 days post-infection. In animals that received the long-acting IL-7 protein at 6 hours post-infection, there was also loss in bodyweight, but the recovery was much quicker compared to the control animals (see, e.g., at day 8 post-infection). And, when the IL-7 protein was administered at 7 days prior to infection, the animals exhibited minimal loss in bodyweight throughout the duration of the experiment.

Similarly, increased survival was observed in animals that received the IL-7 protein prior to the infection (FIG. 1D). When the IL-7 protein was administered at 6 hours post-infection, there was decreased survival compared to animals that received the IL-7 protein prior to the SARS-CoV-2 infection. However, compared to that of the control animals, the survival rate was significantly higher in animals that received the IL-7 protein after infection.

Collectively, the above data demonstrate that the long-acting IL-7 protein described herein can have therapeutic effects and could be useful in treating SARS-CoV-2 infection.

### Example 2: Phase I Study Assessing the Effect of an IL-7 Protein Administration on SARS-CoV-2

A phase I study will be conducted to assess the effect of a long-acting IL-7 protein on SARS-CoV-2 (COVID-19) infection in human subjects. The study will initially test three different doses of the IL-7 protein. Once a safe tolerated dose is established, a double-blind, placebo-controlled randomized pilot study will be conducted to further study the therapeutic efficacy of the IL-7 protein.

### Objective

The primary objective of the phase I study will be to assess the safety and tolerability of the IL-7 protein in patients with COVID-19 infection. The primary objective of the pilot study will be to test the effect of the IL-7 protein on absolute lymphocyte count (ALC) in patients with COVID-19 infection.

Secondary objectives include: (i) to assess the effect of the IL-7 protein on nasopharyngeal, oropharyngeal, or saliva SARS-CoV-2 viral load; (ii) to further assess the safety and tolerability of the IL-7 protein; and (iii) to assess the effect of the IL-7 protein on clinical symptoms and progression of disease using WHO Ordinal Scale.

Additional exploratory objectives include: (i) to evaluate the changes in T lymphocytes subtypes and other PBMC subsets; (ii) to evaluate the changes in cytokine levels; (iii) to explore the association between ALC and viral load; and (iv) to explore the association between ALC and clinical outcome.

### Study Design

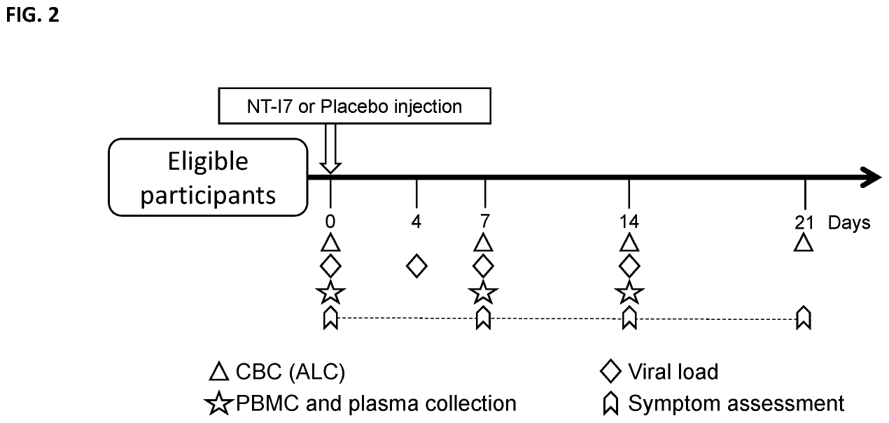

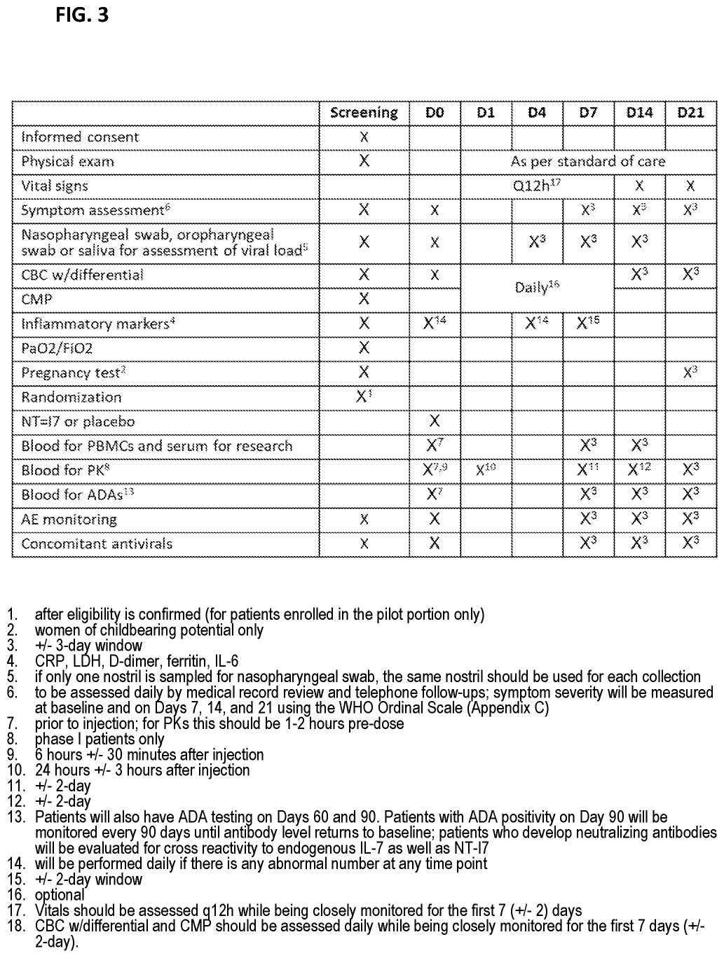

A schematic of the present phase I study is provided in FIG. 2. As shown, eligible subjects will receive a single administration (via intramuscular administration) of either the long-acting IL-7 protein or a placebo (administered within 10 days from symptom onset). Initially, the IL-7 protein will be administered to the subject at a dose of 60 μg/kg (Dose Level 1) and potentially escalate to a dose of 120 μg/kg (Dose Level 2), and if tolerated, then to a dose of 240 μg/kg (Dose Level 3). A cohort of three patients each time will be tested at a dose level and following a strict dose escalation rule (described further below). Staggered dosing will be enacted such that there is a minimum of 72 hours between each patient's dose in a given cohort.

Once a safe tolerated dose is identified from the phase I study, the pilot study will be conducted to test the efficacy of the IL-7 protein. Approximately 30 eligible SARS-CoV-2 positive patients will be stratified by baseline ALC using a cutoff of 1,000 cells/mm³ and double-blind randomized at a 1:1 ratio, and receive either the IL-7 protein (Arm A) or placebo (Arm B). All patients will additionally receive a standard of care treatment (SOC). Toxicity will be closely monitored, and in the presence of unexpected sever adverse events (e.g., death), the study will be suspended immediately. For instance, as shown in FIG. 2, vital signs will be assessed every 12 hours during this period, and blood will be drawn for a CBC with differential (including ALC) and for a CMP prior to injection, daily (at least through Day 7±2 days), and on Days 14 and 21 by regular clinical testing. Inflammatory markers (e.g., d-dimer, ferritin, CRP, LDH) will be drawn prior to injection, on Day 4, and on Day 7 (±2 days). If at any point there is an abnormal value, inflammatory markers will be assessed daily.

Additionally, viral load by PCR will be tested by nasopharyngeal, oropharyngeal or saliva swabs at baseline prior to study treatment, then on Days 4 (optional), 7 and 14 post-injection. If only one nostril will be sampled for nasopharyngeal swabs, the same nostril should be used for each collection. Clinical symptoms will be assessed daily by medical record reviews and telephone follow-ups. Symptom severity will be measured at baseline and on Days 7, 14 and 21 using the WHO Ordinal Scale (see Table 1), and changes will be evaluated from baseline to Days 7, 14 and 21.

Pharmacokinetic (PK) samples will also be collected for all patients in the phase I study at 1-2 hours prior to dosing, 6 hours (±30 minutes) after dosing, 24 hours (±3 hours) after dosing, and on Day 7 (±2 day), Day 14 (±2 day) and Day 21 (±3 day). Additionally, blood for PBMC and serum for research purposes will be collected from all patients (phase I and pilot) prior to injection (on Day 0) and again on Days 7 and 14.

Finally, blood samples will be collected for anti-drug antibody (ADA) analysis for all patients in both phase I and pilot study at baseline, Day 7, Day 14, Day 21, Day 60 and Day 90. Patients with ADA positivity on Day 90 will be monitored every 90 days until antibody level returns to baseline. In addition, patients who develop neutralizing antibodies will be evaluated for cross reactivity to endogenous IL-7, as well as on the therapeutic.

Key inclusion and exclusion criteria for subjects that can be included in the phase I and pilot studies are provided in Table 2 (below).

TABLE 2
Key Inclusion/Exclusion Criteria

| Inclusion | Exclusion |
|---|---|
| Tested PCR positive for SARS-CoV-2by | Receiving any other investigational agents |
| nasopharyngeal swab, oropharyngeal | which may affect patient's lymphocyte |
| swab | counts. Note: There is no evidence that |
| | chloroquine or hydroxychloroquine could |
| | affect lymphocyte counts. Thus, |
| | chloroquine or hydroxychloroquine use is |
| | not an exclusion criteria for this study. |
| | Additionally, it is permissible for potential |
| | participants to have received |
| | investigational or off-label agents for |
| | COVID-19 prior to or during study |
| | participation. |
| Mild COVID-19, defined as WHO | Pregnant or breastfeeding women |
| Ordinal Scale <4 (see Table 1) | |
| Respiratory rate < 20 bpm, HR < 90 bpm, | Transferred from ICU to the floor. |
| and SpO2 > 93% on room air at sea level | |
| Absolute lymphocyte count (ALC) < 1500 | Requiring dialysis. |
| cells/mm3 at the time of screening | |
| Aspartate aminotransferase (AST)/ | Shortness of breath, hypoxia (defined as |
| alanine aminotransferase (ALT) ≤ 3.0 × | PaO2/FiO2 ≤ 300 mmHg), or signs of |
| ULN, total bilirubin ≤ 1.5 × ULN (except | serious lower airway disease. |
| if due to Gilbert's syndrome) | |
| ≥18 years of age | Evidence of acute respiratory distress |
| | syndrome (ARDS), systemic |
| | inflammatory response syndrome |
| | (SIRS)/shock, or cardiac failure. |
| First day of treatment must be no more | Elevated inflammatory markers such as c- |
| than 10 days from onset of COVID-19 | reactive protein (CRP) > 2 × upper limit of |
| symptoms | normal (ULN), lactic acid dehydrogenase |
| | (LDH) > 2 × ULN, D-dimer > 2 × ULN, |
| | ferritin > ULN, and IL-6 > ULN. |
| Individuals of reproductive potential must | Any established diagnosis of autoimmune |
| agree to either abstinence or use of at least | disease requiring systemic treatment |
| one study-approved form of contraception | EXCEPT for vitiligo or endocrine disease |
| when engaging in sexual activities that | (such as diabetes, thyroid disease, and |
| can result in pregnancy from the time of | adrenal disease) controlled by replacement |
| screening through 60 days for female and | therapy. |
| 120 days for male after study agent | |
| administration. Acceptable forms of | |
| contraception for this study are male or | |
| female condoms, diaphragms or cervical | |
| caps with a spermicide, or non-hormonal | |
| intrauterine devices. | |
| Patients with factors or concomitant | Receipt of live attenuated vaccine within |
| illness associated with higher risk of | 30 days before the study treatment. |
| mortality due to COVID-19 (such as older | Examples of live vaccines include, but are |
| age, hypertension, diabetes, and/or | not limited to, the following: measles, |
| COPD) are eligible. | mumps, rubella, chicken pox, yellow |
| | fever, rabies, Bacillus Calmette-Guérin |
| | (BCG), Zoster, and typhoid vaccine. |
| | Seasonal influenza vaccines for injection |
| | are generally killed virus vaccines and are |
| | allowed; however, intranasal influenza |
| | vaccines (e.g. FluMist) are live attenuated |
| | vaccines and are not allowed. |

### Dose Escalation Rule

The overall dose escalation rule is as follows: the study will escalate to the next higher dose level if no DLTs are observed out of a cohort of 3 patients at the current dose level. A total of 6 patients is required at the final safe tolerated dose.

More specifically, three patients will receive the IL-7 protein at Dose Level 1 (i.e., 60 μg/kg). If no DLTs (see Definitions section of the present disclosure) are observed, dosing will escalate to Dose Level 2 (i.e., 120 μg/kg). If one or more DLTs are observed, the study will terminate.

If no DLTs are observed in Dose Level 2, dosing will escalate to Dose Level 3 (i.e., 240 μg/kg). If one or more DLTs are observed in Dose Level 2, dosing will de-escalate to Dose Level 1. If no DLTs are observed in that second cohort of three patients at Dose Level 1, Dose Level 1 is considered the safest tolerated dose. If one or more DLTs are observed in that second cohort of three patients, the study will terminate.

If no DLTs are observed in Dose Level 3, a second cohort of three patients will be enrolled. If no DLTs are observed in that second cohort, Dose Level 3 is considered the safest tolerated dose. If one DLT is observed in either the first or second cohort of patients enrolled to Dose Level 3, dosing will de-escalate to Dose Level 2, and the process will follow the same rule.

### Endpoints

The primary endpoint of the phase I study will be the safe tolerated dose. Toxicities will be summarized by counts and percentages. The primary endpoint of the pilot study will be the ALC percentage change at week 2 from baseline in IL-7 protein treatment arm compared to placebo control arm. The primary endpoint ALC percentage change (% ALC) will be calculated as (week 2 ALC/BL ALC−1)*100%. Summary statistics including mean, median, SD, IQR, range will be provided by arm for ALC at baseline, at week 2 and for % ALC. The % ALC will be compared between the two arms by Wilcoxn rank sum test or two sample t-test as appropriate. Within each arm, the paired ALC changes will be tested against 0 by Wilcoxon signed rank test or paired sample t-test as appropriate.

The secondary endpoints include SARS-CoV-2 viral load PCR test quantitative measurements (if available) and positivity calls at multiple time points (day 0, 7, and 14), toxicity, disease progression indicated by more severe clinical symptoms post treatment in comparison to baseline. The viral load PCR quantitative measurements at each time points will be compared to the baseline within each arm by Wilcoxon signed rank test. The positivity call will be used to calculate the % of patients with a positive PCR test, accompanied with 95% exact binomial CI while Fisher's exact test will be applied to compare the proportions between arms. Adverse events will be graded by CTCAE (version 5.0). Frequency and severity of adverse events will be tabulated using counts and proportions detailing frequently occurring, serious and severe events of interest.

### Outcome Measures

The primary outcome measures of the phase I study include the safety and toleratbility of the IL-7 protein in patients with COVID-19. The primary outcome measures of the pilot study include: ALC increases at week 2 (14 days post treatment) from baseline.

Secondary outcome measures include:
1. Changes of ALC overtime up to 3 weeks post study treatment;
2. Change from baseline to Day 7 in nasopharyngeal, oropharyngeal or saliva SARS-CoV-2 viral load (if quantitative PCR is available); or number of participants by PCR result status (positive or negative) (if quantitative PCR is not available) [Time Frame: Baseline to Day 7]. Viral load assessed by PCR from a nasopharyngeal, oropharyngeal or saliva swab;
3. Change from baseline to Day 14 in nasopharyngeal, oropharyngeal or saliva SARS-CoV-2 viral load (if quantitative PCR is available); or Number of participants by PCR result status (positive or negative) (if quantitative PCR is not available) [Time Frame: Baseline to Day 14]. Viral load assessed by PCR from a nasopharyngeal, oropharyngeal or saliva swab;
4. COVID-19 symptoms severity assessed by WHO Ordinal Scale for clinical improvement [Time Frame: Baseline to Day 7, Day 14 and Day 21];
5. Time to resolution of COVID-19 Symptoms will be assessed daily by WHO Ordinal Scale for clinical improvement [Time Frame: Baseline to Day 21]; and
6. Incidence of treatment-emergent adverse events (TEAE).

### Adverse Events (AEs)

The severity of any AEs will be classified using the CTCAE v5, and will be determined whether the AEs are related or not related to the study treatment (i.e., IL-7 protein administration). Where the AEs are associated with the study treatment (i.e., treatment emergent adverse event (TEAE)), only those events with an onset date prior to date of decision for treatment discontinuation (the later of the date of the decision of the Investigator to permanently discontinue study treatment or the date of the last dose of study treatment taken by the subject)+30 days (+100 days for SAEs and certain other AEs) will be considered and tabulated.

The frequency and percentage of subjects with TEAEs will be tabulated for overall incidence by system organ class and/or preferred term by treatment arm. Related TEAEs, serious TEAEs, related serious TEAEs, high-grade TEAEs, Grade 5 TEAEs, and TEAEs resulting in study treatment discontinuation will be similarly summarized. Summaries by worst reported severity for each event within a subject will also be provided.

### Example 3: Analysis of the Effect of IL-7 Protein Administration on SARS-CoV-2 Delta Variant

To further assess effect on other coronaviruses, the therapeutic effects of long-acting IL-7 protein (e.g., described herein) on the delta variant of SARS-CoV-2 will be assessed. For instance, a suitable animal model (e.g., mice described in Example 1) will be infected with the SARS-CoV-2 variant (e.g., NCCP 43390 (hCoV-19/Korea119861/KDCA/2021) from the National Culture Collection for pathogens of Korea Centers for Disease Control and Prevention (KCDC)). The IL-7 protein will be administered to the animals at various time points: (1) prior to the infection; (2) after the infection; or (3) both prior and after the infection. Then, the therapeutic effects of the IL-7 protein will be assessed in the animals using any suitable methods known in the art (e.g., bodyweight loss and/or survival as described in Example 1).

### Example 4: Analysis of the Effect of IL-7 Protein Administration on SARS-CoV-2 Gamma Variant

To further assess effect on other coronaviruses, the therapeutic effects of long-acting IL-7 protein (e.g., described herein) on the gamma variant of SARS-CoV-2 will be assessed. For instance, a suitable animal model (e.g., mice described in Example 1) will be infected with the SARS-CoV-2 variant (e.g., NCCP 43388 (hCoV-19/Korea/KDCA95637/2021) from the National Culture Collection for pathogens of Korea Centers for Disease Control and Prevention (KCDC)). The TL-7 protein will be administered to the animals at various time points: (1) prior to the infection; (2) after the infection; or (3) both prior and after the infection. Then, the therapeutic effects of the TL-7 protein will be assessed in the animals using any suitable methods known in the art (e.g., bodyweight loss and/or survival as described in Example 1).

1. A method of treating a disease or disorder associated with a coronavirus infection in a subject in need thereof, comprising administering to the subject an interleukin-7 (IL-7) fusion protein, wherein the IL-7 fusion protein comprises an IL-7 protein and a half-life extending moiety.

2. The method of claim 1, wherein: (a) the coronavirus infection is associated with (1) a mild disease as defined by the WHO Ordinal Scale (i.e., <4) or (2) a severe disease as defined by the WHO Ordinal Scale (i.e., 5-7): (b) the subject does not exhibit severe hypoxic respiratory failure; or (c) both (a) and (b).

3-4. (canceled)

5. The method of claim 1, wherein after the administration: (a) an absolute lymphocyte count (ALC) is increased in the subject; (b) a total number of an immune cell is increased in the subject (c) a viral load is decreased in the subject: or (d) any combination thereof.

6-8. (canceled)

9. The method of claim 5, wherein the immune cell comprises a CD4⁺ T cell, CD8⁺ T cell, NK cell, B cell, mucosal associated invariant T (MAIT) cell, innate lymphoid cells (ILCs), or combinations thereof.

10-11. (canceled)

12. A method of treating and/or reducing a lymphopenia in a subject suffering from a coronavirus infection, comprising administering to the subject an interleukin-7 (IL-7) fusion protein, wherein the IL-7 fusion protein comprises an IL-7 protein and a half-life extending moiety.

13. The method of claim 12, wherein: (a) the coronavirus infection is associated with (1) a mild disease as defined by the WHO Ordinal Scale (i.e., <4) or (2) a severe disease as defined by the WHO Ordinal Scale (i.e., 5-7); (b) the subject does not exhibit a severe hypoxic respiratory failure; or (c) both (a) and (b).

14-15. (canceled)

16. The method ofany one of claim 12, wherein the lymphopenia is characterized by: (a) a total lymphocyte count that is less than by at least about 5% compared to a corresponding value in a reference subject (e.g., subject who does not have a coronavirus infection); (b) a circulating blood total lymphocyte count of less than at least about 50 lymphocytes/uL; or (c) both (a) and (b).

17-21. (canceled)

22. The method of claim 12, wherein after the administration: (a) the total lymphocyte count in the subject is increased, (b) the number of CD4⁺ T cells in the subject is increased, (c) the number of CD8⁺ T cells in the subject is increased, or (d) any combination of (a) to (c).

23-25. (canceled)

26. A method of increasing a survival of an immune cell in a subject suffering from a coronavirus infection, comprising administering to the subject an interleukin-7 (IL-7) fusion protein, wherein the IL-7 fusion protein comprises an IL-7 protein and a half-life extending moiety.

27. The method of claim 26, wherein: (a) the coronavirus infection is associated with (1) a mild disease as defined by the WHO Ordinal Scale (i.e., <4) or (2) a severe disease as defined by the WHO Ordinal Scale (i.e., 5-7): (b) the subject does not exhibit a severe hypoxic respiratory failure; or (c) both (a) and (b).

28-32. (canceled)

33. The method of claim 26, wherein the immune cell comprises a CD4+ T cell, CD8+ T cell, NK cell, B cell, mucosal associated invariant T (MAIT) cell, innate lymphoid cells (ILCs), or combinations thereof.

34-35. (canceled)

36. The method of claim 1, further comprising administering one or more additional therapeutic agents to the subject.

37-38. (canceled)

39. The method of claim 36, wherein the one or more additional therapeutic agents comprise a standard care of treatment, antiprotozoal agent (e.g., chloroquine or hydroxychloroquine (with or without azithromycin)), antiparasitic agent (e.g., ivermectin), antibiotic agent (e.g., azithromycin), protease inhibitor (e.g., lopinavir/ritonavir or darunavir/cobicistat), immune-based therapy, adjunctive therapy (e.g., antithrombotic therapy), vitamins (e.g., vitamin C (ascorbic acid) and vitamin D), zinc supplementation, or combinations thereof.

40-43. (canceled)

44. The method of claim 1, wherein the coronavirus comprises a SARS-CoV-1, SARS-CoV-2 (COVID-19), MERS-CoV, including mutants and variants thereof, or combinations thereof.

45. The method of claim 1, wherein the IL-7 fusion protein is administered: (a) at a dose between about 20 μg/kg and about 600 μg/kg or at a dose between about 600 μg/kg to about 2,000 μg/kg: (b) at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks: or (c) both (a) and (b).

46-54. (canceled)

55. The method of claim 1, wherein the IL-7 fusion protein further comprises an oligopeptide consisting of 1 to 10 amino acid residues.

56. (canceled)

57. The method of claim 55, wherein: (a) the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof; (b) the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof; or (c) both (a) and (b).

58-63. (canceled)

64. The method of claim 1, wherein the IL-7 protein comprises an amino acid sequence having a sequence identity of at least about 70% to the amino acid sequence set forth in any one of SEQ ID NOs: 1-6 and 15-25.

65. The method of claim 1, wherein the IL-7 protein does not comprise amino acid residues 1-25 of the amino acid sequence set forth in any one of SEQ ID NOs: 1-6.

66. The method of claim 1, wherein the IL-7 protein comprises: (i) amino acid residues 26-178 of SEQ ID NO: 1, (ii) amino acid residues 26-155 of SEQ ID NO: 2, (iii) amino acid residues 26-155 of SEQ ID NO: 3, (iv) amino acid residues 26-178 of SEQ ID NO: 4, (v) amino acid residues 26-177 of SEQ ID NO: 5, or (iv) amino acid residues 26-177 of SEQ ID NO: 6.


SEQUENCE LISTING (72 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, Rattus norvegicus
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, Mus Musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, Cercopithecus aethiops
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, Bos taurus
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, Ovis aries
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, Artificial Sequence
  amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, Artificial Sequence
  amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, Artificial Sequence
  amino acid sequence of hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, Artificial Sequence
  amino acid sequence of hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, Artificial Sequence
  amino acid sequence of hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, Artificial Sequence
  amino acid sequence of hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, Artificial Sequence
  amino acid sequence of hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, Artificial Sequence
  amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 153 aa, Artificial Sequence
  amino acid sequence of modified IL-7(M)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 154 aa, Artificial Sequence
  amino acid sequence of modified IL-7(MM)
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 155 aa, Artificial Sequence
  amino acid sequence of modified IL-7(MMM)
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 155 aa, Artificial Sequence
  amino acid sequence of modified IL-7(MGM)
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 155 aa, Artificial Sequence
  amino acid sequence of modified IL-7(DDD)
DDDDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 156 aa, Artificial Sequence
  amino acid sequence of modified IL-7(MMMM)
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 398 aa, Artificial Sequence
  amino acid sequence of modified IL-7(M) fused hyFc
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 399 aa, Artificial Sequence
  amino acid sequence of modified IL-7(MM) fused hyFc
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 400 aa, Artificial Sequence
  amino acid sequence of modified IL-7(MMM) fused hyFc
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 400 aa, Artificial Sequence
  amino acid sequence of modified IL-7(MGM) fused hyFc
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 401 aa, Artificial Sequence
  amino acid sequence of modified IL-7(MMMM) fused hyFc
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 397 aa, Artificial Sequence
  amino acid sequence of human IL-7 fused hyFc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 395 aa, Artificial Sequence
  amino acid sequence of human IL-7 fused nonlytic mouse Fc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 28 - DNA, 531 bp, Artificial Sequence
  nucleotide sequence of human IL-7
atgttccacgtgagcttcaggtacatcttcggcctgccacccctgatcctggtgctgctgcctgtggccagctccgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacac

SEQ ID NO: 29 - DNA, 534 bp, Artificial Sequence
  nucleotide sequence of modified IL-7(M)
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 30 - DNA, 537 bp, Artificial Sequence
  nucleotide sequence of modified IL-7(MM)
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 31 - DNA, 540 bp, Artificial Sequence
  nucleotide sequence of modified IL-7(MMM)
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 32 - DNA, 540 bp, Artificial Sequence
  nucleotide sequence of modified IL-7(MGM)
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcaccaaggagcac

SEQ ID NO: 33 - DNA, 540 bp, Artificial Sequence
  nucleotide sequence of modified IL-7(DDD)
atgttccacgtgagcttcagatacatcttcggcctgccccccctgatcctggtgctgctgcccgtggccagcagcgacgatgacgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 34 - DNA, 543 bp, Artificial Sequence
  nucleotide sequence of modified IL-7(MMMM)
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 35 - DNA, 1284 bp, Artificial Sequence
  nucleotide sequence of modified IL-7(M) fused hyFc
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtgactcgagtctaga

SEQ ID NO: 36 - DNA, 1272 bp, Artificial Sequence
  nucleotide sequence of modified IL-7(MM) fused hyFc
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 37 - DNA, 1275 bp, Artificial Sequence
  nucleotide sequence of modified IL-7(MMM) fused hyFc
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 38 - DNA, 1275 bp, Artificial Sequence
  nucleotide sequence of modified IL-7(MGM) fused hyFc
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 39 - DNA, 1278 bp, Artificial Sequence
  nucleotide sequence of modified IL-7(MMMM) fused hyFc
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 40 - protein, 4 aa, Artificial Sequence
  synthetic oligopeptide fragment
MMMM

SEQ ID NO: 41 - protein, 4 aa, Artificial Sequence
  synthetic oligopeptide fragment
MGGM

SEQ ID NO: 42 - protein, 4 aa, Artificial Sequence
  synthetic oligopeptide fragment
MMGG

SEQ ID NO: 43 - protein, 4 aa, Artificial Sequence
  synthetic oligopeptide fragment
GGMM

SEQ ID NO: 44 - protein, 4 aa, Artificial Sequence
  synthetic oligopeptide fragment
MGMG

SEQ ID NO: 45 - protein, 4 aa, Artificial Sequence
  synthetic oligopeptide fragment
GMMG

SEQ ID NO: 46 - protein, 4 aa, Artificial Sequence
  synthetic oligopeptide fragment
GGGM

SEQ ID NO: 47 - protein, 4 aa, Artificial Sequence
  synthetic oligopeptide fragment
MGGG

SEQ ID NO: 48 - protein, 4 aa, Artificial Sequence
  synthetic oligopeptide fragment
GMGG

SEQ ID NO: 49 - protein, 4 aa, Artificial Sequence
  synthetic oligopeptide fragment
GGMG

SEQ ID NO: 50 - protein, 5 aa, Artificial Sequence
  synthetic oligopeptide fragment
GGMMM

SEQ ID NO: 51 - protein, 5 aa, Artificial Sequence
  synthetic oligopeptide fragment
GGGMM

SEQ ID NO: 52 - protein, 5 aa, Artificial Sequence
  synthetic oligopeptide fragment
GGGGM

SEQ ID NO: 53 - protein, 5 aa, Artificial Sequence
  synthetic oligopeptide fragment
MGMMM

SEQ ID NO: 54 - protein, 5 aa, Artificial Sequence
  synthetic oligopeptide fragment
MGGMM

SEQ ID NO: 55 - protein, 5 aa, Artificial Sequence
  synthetic oligopeptide fragment
MGGGM

SEQ ID NO: 56 - protein, 5 aa, Artificial Sequence
  synthetic oligopeptide fragment
MMGMM

SEQ ID NO: 57 - protein, 5 aa, Artificial Sequence
  synthetic oligopeptide fragment
MMGGM

SEQ ID NO: 58 - protein, 5 aa, Artificial Sequence
  synthetic oligopeptide fragment
MMGGG

SEQ ID NO: 59 - protein, 5 aa, Artificial Sequence
  synthetic oligopeptide fragment
MMMGM

SEQ ID NO: 60 - protein, 5 aa, Artificial Sequence
  synthetic oligopeptide fragment
MGMGM

SEQ ID NO: 61 - protein, 5 aa, Artificial Sequence
  synthetic oligopeptide fragment
GMGMG

SEQ ID NO: 62 - protein, 5 aa, Artificial Sequence
  synthetic oligopeptide fragment
GMMMG

SEQ ID NO: 63 - protein, 5 aa, Artificial Sequence
  synthetic oligopeptide fragment
GGMGM

SEQ ID NO: 64 - protein, 5 aa, Artificial Sequence
  synthetic oligopeptide fragment
GGMMG

SEQ ID NO: 65 - protein, 5 aa, Artificial Sequence
  synthetic oligopeptide fragment
GMMGM

SEQ ID NO: 66 - protein, 5 aa, Artificial Sequence
  synthetic oligopeptide fragment
MGMMG

SEQ ID NO: 67 - protein, 5 aa, Artificial Sequence
  synthetic oligopeptide fragment
GMGGM

SEQ ID NO: 68 - protein, 5 aa, Artificial Sequence
  synthetic oligopeptide fragment
MMGMG

SEQ ID NO: 69 - protein, 5 aa, Artificial Sequence
  synthetic oligopeptide fragment
GMMGG

SEQ ID NO: 70 - protein, 5 aa, Artificial Sequence
  synthetic oligopeptide fragment
GMGGG

SEQ ID NO: 71 - protein, 5 aa, Artificial Sequence
  synthetic oligopeptide fragment
GGMGG

SEQ ID NO: 72 - protein, 5 aa, Artificial Sequence
  synthetic oligopeptide fragment
GGGGG